(19) United States
(12) Patent Application Publication
(10) Pub. No.: US 2025/0366761 A1
(43) Pub. Date: Dec. 4, 2025

(54) Bidirectional Optical, Electrical, and Chemical Neural Probe

(71) Applicant: Massachusetts Institute of Technology, Cambridge, MA (US)

(72) Inventors: Polina Olegovna ANIKEEVA, Lexington, MA (US); Marc-Joseph Antonini, Somerville, MA (US); Atharva Sahasrabudhe, Cambridge, MA (US); Nicolette Driscoll, Somerville, MA (US)

(73) Assignee: Massachusetts Institute of Technology, Cambridge, MA (US)

(21) Appl. No.: 19/221,073

(22) Filed: May 28, 2025

Related U.S. Application Data
(60) Provisional application No. 63/652,441, filed on May 28, 2024.

(51) Int. Cl. A61B 5/294; A61B 5/1473; A61B 5/263

(52) U.S. Cl. CPC A61B 5/294; A61B 5/1473; A61B 5/263; A61B 2562/125

(57) ABSTRACT

A multifunctional fiber probe capable of optical, electrical, and chemical interrogation of neuronal brain circuits in vivo is disclosed. The fiber probe may include an optical waveguide to enable optogenetic stimulation and/or fiber photometry, one or more electrodes for electrical recording and/or stimulation, and a microfluidic conduit to deliver at a drug, a gene, and/or a chemical to a mammalian subject. The fiber probe may be MRI compatible. The fiber probe may have a minimal footprint and enhanced bio-compatibility due to its flexible materials, resulting in reduced inflammation relative to conventional deep brain stimulation probes. The fiber probe may allow for multisite optical, electrical, and viral perturbations and electrophysiological and photometric recordings.

### CROSS-REFERENCE TO RELATED APPLICATIONS

The present application claims the priority benefit, under 35 U.S.C. 119 (e), of U.S. Application No. 63/652,441, filed on May 28, 2024, which is incorporated herein by reference in its entirety for all purposes.

### GOVERNMENT SUPPORT

This invention was made with government support under EEC1028725 awarded by the National Science Foundation. The government has certain rights in the invention.

### BACKGROUND

Understanding neurophysiological phenomena underlying complex neurological diseases and disorders demands tools capable of recording and modulating a diversity of neural signals. Furthermore, as many disorders of the nervous system emerge over extended periods of time, the ability to monitor neural dynamics over chronic time periods relies on the biocompatibility and reliable performance of the neural interfacing device for periods ranging from minutes to years. In this context, the integration of an increasing number of functional features to probe neural circuit complexity is at odds with the desire to reduce the footprint and mechanical stiffness of devices to minimize the tissue damage and ensure long-term biocompatibility and device functionality. Some examples of tools used to enable the biological discovery of causal neural circuits include fiber photometry, optogenetics, electrical stimulation, and recordings. Advances in genetic and optical engineering have also allowed scientists to probe these pathways with cell-type and temporal specificity, to uncover mechanisms critical to the understanding of health and disease.

However, the combination of these technologies with other preclinical techniques, such as magnetic resonance imaging (MRI), has remained limited. MRI can be paired with electrical deep brain stimulation (DBS) to study neural circuitry and patterns of brain activation. Notwithstanding, local magnetic field inhomogeneities that result from the implantation of traditional silver bipolar electrodes impede signal collection, particularly in the area surrounding the device. Single-shot echo-planar imaging (ssEPI) may be used for functional MRI imaging due to its high signal-to-noise ratio (SNR), rapid acquisition, and insensitivity to motion. However, this imaging technique is particularly sensitive to susceptibility-induced field inhomogeneities and low sampling bandwidth along the phase-encoding direction. As a result, data from ssEPI often suffers from low SNR when using conventional metallic electrodes for DBS during MRI imaging. Similarly, fast-scan cyclic voltammetry (FSCV) can be used to measure the release of catecholamines at high sampling rates, revealing real-time dopamine (DA) release dynamics. Nevertheless, the use of FSCV in vivo has been limited by the decreased sensitivity of the electrodes caused by biofouling once the probes are implanted into a brain.

### SUMMARY

To circumvent these challenges, disclosed herein is a bidirectional multifunctional fiber probe (referred to herein as a Polymer-based Optical-electrical-chemical neuroLogical Interface (POLI) fiber probe or POLI fiber) using carbon nanotube (CNT) electrodes that not only demonstrates exceptional electrical impedance properties for electrophysiological recording, but also has a low magnetic susceptibility, making it ideal for MRI experiments. Furthermore, the micron-scale coarse texture on the surface of the CNTs may act as a DA trap which increases both the conductivity and sensitivity at the surface of the electrode for FSCV measurements. The micron-scale coarse texture may be present on macroscale materials made from CNT fiber and/or CNT yarn that have been assembled into a larger form facture. To improve the biocompatibility of the POLI fiber and reduce its footprint in the brain, a flexible PMMA/THVP waveguide formulation transmits light comparable to standard silica waveguides. These electrical and optical functionalities, in addition to a fluidics channel, are combined into a single fiber using thermal drawing. In brief, a centimeter scale preform, containing all desired POLI fiber components, is heated and drawn into a microscale fiber that retains the cross-sectional geometry of the original model.

The POLI fiber probe disclosed herein may also overcome previous limitations of fiber-based neurotechnologies through its material choice and fabrication techniques. The POLI fiber probe is an MRI-compatible technology that combines the capabilities of electrophysiology, chemical sensing, optogenetics, and photometry into a footprint smaller than a current single optical waveguides, while also improving vector targeting through the integrated fluidics channel. The POLI fiber probe disclosed herein may be applied to study multivariate features of the mesolimbic reward in parallel.

The multimaterial fiber technology of the POLI fiber probe enables a straightforward integration of optical, electrophysiological, and microfluidic capabilities within miniature and compliant neural interfaces. The POLI fiber probe may enable simultaneous recording, optogenetic stimulation, and drug and gene delivery in the brain of freely moving mice. The flexible POLI fiber probes may also impart minimal damage to the local tissues as evidenced by their ability to track isolated neuronal potentials over 6 months, as well as by the negligible glial scar formation in their vicinity. Additionally, because the POLI fiber probes may be based on polymers, composites, and non-magnetic metals, they may be compatible with magnetic resonance imaging (MRI), exhibiting negligible shadow artifacts even in high-Tesla MRI scanners. This suggests the utility of these POLI fiber probes for correlating local recording or manipulations of neural circuit function to brain-wide mapping of neural states.

Herein the capabilities of the POLI fiber probes are also expanded to permit electrical stimulation, photometric readout of fluorescent neural activity indicators, and fast-scan cyclic voltammetry (FSCV) sensing of neurochemicals. These capabilities may be valuable in studies of neural circuits and for the development of clinically-translatable neuromodulation approaches for the treatment of brain disorders. Until now, these functions have never been integrated within a single neural probe compatible with long-term behavioral studies.

In some aspects, the techniques described herein relate to a multifunctional fiber including a microfluidic conduit to deliver at least one of a drug, a gene, or a chemical to a mammalian subject, at least one electrode to enable at least one of electrical recording of neural activity, electrochemical recording of neural activity, or electrical stimulation of neural activity in the mammalian subject, and an optical waveguide to enable at least one of optogenetic stimulation or fiber photometry, wherein the microfluidic conduit is peelable from the multifunctional fiber.

In some aspects, the techniques described herein relate to a multifunctional fiber wherein the optical waveguide includes at least two polymers and wherein the at least two polymers include at least one of poly(methyl-methacrylate) (PMMA)/Cyclic olefin copolymer (COC), PMMA/Polycarbonate (PC), or PMMA/tetrafluoroethylene hexafluoropropylene, vinylidene fluoride (THVP).

In some aspects, the techniques described herein relate to a multifunctional fiber wherein the optical waveguide includes a core including poly(methyl-methacrylate) and a cladding including tetrafluoroethylene, hexafluoropropylene, and vinylidene fluoride.

In some aspects, the techniques described herein relate to a multifunctional fiber wherein the multifunctional fiber is formed from thermal drawing.

In some aspects, the techniques described herein relate to a multifunctional fiber wherein the multifunctional fiber further includes at least one layer of an elastomer between the microfluidic conduit and the multifunctional fiber.

In some aspects, the techniques described herein relate to a multifunctional fiber wherein the at least one electrode enables the electrical recording of neural activity, the electrochemical recording of neural activity, and the electrical stimulation of neural activity in the mammalian subject concurrently.

In some aspects, the techniques described herein relate to a multifunctional fiber wherein the electrochemical recording of neural activity is a chemical recording of a neurotransmitter using fast-scan cyclic voltammetry.

In some aspects, the techniques described herein relate to a multifunctional fiber wherein the neurotransmitter is dopamine.

In some aspects, the techniques described herein relate to a multifunctional fiber wherein the at least one electrode has a charge injection capacity of about 5 mC/cm2 to about 30 mC/cm2.

In some aspects, the techniques described herein relate to a multifunctional fiber wherein the at least one electrode has a cathodic charge storage capacity of about 5000 mC/cm2 to about 9000 mC/cm2.

In some aspects, the techniques described herein relate to a multifunctional fiber wherein the electrical stimulation is deep brain stimulation.

In some aspects, the techniques described herein relate to a multifunctional fiber wherein the at least one electrode includes at least one of a carbon nanotube (CNT) fiber, a tungsten (W) microwire, or a conductive microwire.

In some aspects, the techniques described herein relate to a multifunctional fiber wherein an end of the microfluidic conduit is peeled back from the multifunctional fiber and further comprising a tube coupled to the end of the microfluidic conduit.

In some aspects, the techniques described herein relate to a multifunctional fiber wherein the end of the microfluidic conduit is inserted into a lumen of the tube.

In some aspects, the techniques described herein relate to a multifunctional fiber wherein the tube is mechanically connected to the multifunctional fiber with an epoxy.

In some aspects, the techniques described herein relate to an assembly including the multifunctional fiber, the assembly further including a housing to hold a proximal end of the multifunctional fiber, wherein a distal end of the fiber is configured to be interested into tissue and a proximal end of the microfluidic conduit is peeled back from the multifunctional fiber, a tube coupled to the proximal end of the microfluidic conduit, a printed circuit board operably connected to the at least one electrode, and an optical ferrule operably connected to the optical waveguide.

In some aspects, the techniques described herein relate to a method of making a multifunctional fiber probe assembly, the method including forming a polymer preform defining at least a microfluidic channel, an optical waveguide channel, and an electrode channel, thermally drawing the polymer preform to form a polymer fiber, converging at least one electrode microwire through the electrode channel, and mechanically separating the microfluidic channel from the polymer fiber.

In some aspects, the techniques described herein relate to a method, wherein forming the polymer preform defining the microfluidic channel further includes forming a first polymer wall around the microfluidic channel and forming a second polymer layer around the first polymer wall, wherein the second polymer layer has a weak adhesion to the first polymer wall.

In some aspects, the techniques described herein relate to a method, further includes inserting the microfluidic channel into a lumen of a tube and sealing the tube to the polymer fiber with an epoxy.

In some aspects, the techniques described herein relate to a method, further including exposing a distal portion of the electrode microwire running through the electrode channel, electrically coupling the distal portion of the electrode microwire to a printed circuit board, mechanically coupling the optical waveguide channel to an optical ferrule, and inserting a proximal end of the polymer fiber into a housing.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A shows a schematic of the thermal drawing process with carbon nanotube (CNT) microwires converged into the fiber (left panel) and schematic of cross-section of an Polymer-based Optical-electrical-chemical neuroLogical Interface (POLI) fiber probe with different functional components labelled (right panel pop out).

FIG. 1B shows a photograph of the multi-material preform of the POLI fiber probe.

FIG. 1C shows a cross-section of the POLI fiber probe after thermal drawing. The probe is approximately 300 μm×280 μm.

FIG. 2 shows a diagram of the peelable micro-fluidic (μfluidic) channel connectorization. The channel is peeled away from the bulk of the fiber (left panel), inserted into the lumen of a larger-diameter flexible tubing (middle panel), then epoxied in place (right panel).

FIG. 3A shows a schematic of experimental recording and stimulation paradigm in a DAT-Cre mouse line. AAVs to deliver the optical dopamine reporter dLight1.1 to the nucleus accumbens (Nac) and the red excitatory opsin ChrimsonR to the ventral tegmental area (VTA) were injected through the microfluidic channel in the fiber probe during implantation.

FIG. 3B shows an electrophysiology recording of optogenetically-evoked activity of dopamine (DA) neurons in VTA.

FIG. 3C shows a fiber photometry recording of DA transients in NAc (dLight1.1) evoked by electrical stimulation in VTA.

FIG. 3D shows a fiber photometry recording of DA transients in NAc before and after intraperitoneal (IP) administration of cocaine (about 20 mg/kg) evoked by electrical stimulation in NAc. A slower DA reuptake is observed in the presence of cocaine (upper trace).

FIG. 4A shows a schematic of the cross-section of an example POLI fiber, highlighting its recording and stimulating capabilities.

FIG. 4B shows a diagram of the convergence drawing process used to fabricate the POLI fiber.

FIG. 4C shows a photograph of the macroscale preform highlighting a polymer combination choice and the channels used for electrodes and microfluidics.

FIG. 4D shows an optical micrograph of the POLI fiber cross-section highlighting the cross-sectional geometry and embedded carbon nanotube (CNT) microwires.

FIG. 4E shows a photograph highlighting the flexibility and microscopic size of the POLI fiber.

FIG. 4F shows a fully assembled POLI fiber with electrical pins, optical ferrule, and a microfluidic inlet.

FIG. 4G shows a photograph of a dual-implant assembly that leverages a printed circuit board (PCB) and an Omnetics connector to facilitate connectorization. This implant may simplify the implantation process of fiber devices targeting the VTA and NAc.

FIG. 4H shows a photograph of a freely moving mouse with the dual-fiber implant shown in FIG. 4G.

FIG. 5A shows a picture of a poly(methyl methacrylate) (PMMA)/terpolymer of tetrafluoroethylene, hexafluoropropylene, and vinylidene fluoride (THVP) waveguide implanted in a fluorescein-doped agarose brain transmitting blue (e.g., about 470 nm) light to elicit a fluorescent response (approximately 520 nm).

FIG. 5B shows a graph of the evaluation of optical loss for 400 μm polycarbonate (PC)/PMMA, cyclic-olefin-copolymer (COC)/PMMA, and PMMA/THVP polymer fibers, as well as the 200 μm PMMA/THVP optical waveguide of the POLI Fiber. Optical loss described as mean decibel loss±s.e.m. (n=3 samples).

FIG. 5C shows numerical aperture measurements of 400 μm PC/PMMA (middle), COC/PMMA (left), and PMMA/THVP (right) polymer fibers, expressed as mean±s.e.m. (n=3 samples).

FIG. 5D shows a Bode plot of impedance magnitude and phase of the POLI fiber-embedded 20 μm-diameter CNT microelectrodes (n=3 fibers, 18 electrodes total).

FIG. 5E shows cyclic voltammograms of the CNT electrodes and same-size comparison stainless-steel electrodes in phosphate-buffered saline (PBS) and their respective cathodic charge storage capacity values.

FIG. 5F shows a voltage transient response of CNT electrodes to symmetric, biphasic, charge-balanced, current pulses of 250 usec half phase and a 250 μsec interphase delay, and corresponding cathodic charge injection capacity.

FIG. 5G shows fast scan cyclic voltammetry response of a CNT electrode to a bolus of 20 μM dopamine (DA) solution delivered at t=7 seconds recorded in a flow cell.

FIG. 5H shows a representative voltammogram of CNT electrode response to 20 μM DA solution, showing DA oxidation peak at +0.4V and reduction peak at 0V.

FIG. 5I shows a DA calibration curve demonstrating a linear relationship between detected current and dopamine concentration in solution (R²=0.994, N=3).

FIG. 5J shows current measured at +0.4 V vs. time for N=5 trials of 20 μM DA solution bolus delivered in the flow cell, converted to DA concentration using the calibration curve shown in FIG. 5I.

FIG. 5K shows a graph of the measured rate of fluid injection via POLI fiber microfluidic channel vs. rate set on the infusion pump. Infusions were delivered at set rates between 25, 50, and 75 nl/minute and are represented as mean±s.e.m. (n=3).

FIG. 5L shows a graph of stiffness measured via dynamic mechanical analysis of POLI fibers (n=3) compared to a similarly sized silica fiber across the frequency ranges of locomotion, respiration, and heart rate.

FIG. 6A shows a schematic of a POLI fiber implanted in the whisker sensory cortices (SIBF) of Thy1-GCaMP6s mice and used to concurrently stimulate electrically through the embedded CNT electrodes and record stimulation evoked calcium influxes using fiber photometry.

FIG. 6B shows calcium transient measured following an electrical stimulation train of 10 Hz, with current varying between 35-215 μA.

FIG. 6C shows a graph of mean fluorescence (ΔF/F₀)±s.e.m, with fluorescence normalized to the max fluorescence value following an electrical stimulation train of 10 Hz.

FIG. 6D shows calcium transient measured following an electrical stimulation train of 130 Hz with current varying between 35-215 μA.

FIG. 6E shows a graph showing fluorescence represented as a mean fluorescence (ΔF/F₀)±s.e.m, with fluorescence normalized to the max fluorescence value following an electrical stimulation train of 130 Hz with current varying between 35-215 μA.

FIG. 6F shows a schematic of high field MRI acquisition, which was performed with a 20 cm-bore 9.4 T Bruker small animal scanner with a custom-made 30 mm single surface coil used as a transceiver.

FIG. 6G shows an anatomical scan acquired using a T2-weighted rapid acquisition with refocused echoes pulse sequence for the POLI fiber.

FIG. 6H shows an anatomical scan acquired using a T2-weighted rapid acquisition with refocused echoes pulse sequence for a silver electrode.

FIG. 6I shows a functional scan performed using T2*-weighted EPI sequence for detection of stimulus-induced BOLD contrast for the POLI fiber.

FIG. 6J shows a functional scan performed using T2*-weighted EPI sequence for detection of stimulus-induced BOLD contrast for a silver electrode.

FIG. 6K shows a scan of deep brain stimulation of 0.1 mA for 2 seconds at a frequency of 60 Hz, which was preceded by a 10-second baseline scan and followed by a 48-second recovery scan, repeated over 30 cycles for the POLI fiber.

FIG. 6L shows a scan of deep brain stimulation of 0.1 mA for 2 seconds at a frequency of 60 Hz, which was preceded by a 10-second baseline scan and followed by a 48-second recovery scan, repeated over 30 cycles for a silver electrode.

FIG. 6M shows a graph of the preprocessing of the functional scans aligned to high-resolution anatomical images showing the number of voxels of the MRI image that show implant-related artifact or shadow for the POLI fiber (left bar) versus silver electrodes (right bar). To quantify voxel count, a threshold of SNR<5 was applied on slices in which the implant was observed. ** corresponds to P<0.01.

FIG. 6N shows a graph of maximum signal amplitude during an interval of 6 seconds after stimulation onset was measured and compared the average preceding baseline interval via student-t-test to evaluate statistical significance of Z-values. To quantify voxel count, a threshold of SNR<5 was applied on slices in which the implant was observed. ** corresponds to P<0.01. The POLI fiber (left bar) produces a greater increase in BOLD signal compared to identical stimulation delivered via silver electrodes (right bar).

FIG. 6O shows a schematic of fast-scan cyclic voltammetry (FSCV) performed through POLI fiber used to detect DA release in NAc in response to electrical stimulation in VTA supplied via an electrode.

FIG. 6P shows a representative plot for the FSCV from FIG. 6O.

FIG. 6Q shows a graph of voltage transient for the FSCV from FIG. 6O.

FIG. 6R shows a representative voltammogram for FSCV from FIG. 6O. The DA concentration was calculated from maximum at its oxidation peak in the representative voltammogram.

FIG. 7A shows a schematic of a mouse brain implanted with the dual implant device, comprised of two POLI fibers targeting the NAc and the VTA, and transfected with DA fluorescent indicator dLight1.1 into the NAc via AAV delivery through the POLI fiber fluidic channel.

FIG. 7B shows the endogenous electrophysiology activity recorded in VTA, showing multiunit neural activity with phasic and tonic firing. Dots indicate spikes detected for sorting. Shaded areas indicate phasic firing interspersed with tonic firing in unshaded areas.

FIG. 7C shows fiber photometry recording of endogenous DA dynamics in NAc concomitant with recordings in FIG. 7B. Shaded areas indicate phasic firing interspersed with tonic firing in unshaded areas. Large DA transients observed in NAc photometry recordings are time-locked to bursts of tonic firing of the neurons in VTA.

FIG. 7D shows a principal components analysis and clustering of neural spike waveforms recorded in VTA for the two neuronal units.

FIG. 7E shows mean spike waveforms for the two neuronal units.

FIG. 7F shows a schematic of the device. Mice were implanted with the dual implant POLI fiber device and transfected with a red excitatory opsin ChrimsonR in the VTA via AAV9 delivery through the POLI fiber channel to enable simultaneous optogenetic stimulation and electrophysiology recording.

FIG. 7G shows the optogenetic stimulation-evoked activity of VTA neurons with 5 Hz stimulation.

FIG. 7H shows the optogenetic stimulation-evoked activity of VTA neurons with 20 Hz stimulation.

FIG. 7I shows a fluorescent image showing the expression of dLight1.1 in the NAc.

FIG. 7J shows a fluorescent image showing the expression of ChrimsonR in the VTA.

FIG. 7K shows a schematic of the experiment. In the same cohort of animals used in FIGS. 7A-7E, electrical stimulation was applied either to the VTA or to the NAc while recording DA dynamics in NAc via fiber photometry.

FIG. 7L shows electrical stimulation of DA axon terminals in the NAc produced robust DA transients recorded via photometry. Stimulation epochs were recorded before and after intraperitoneal (IP) administration of cocaine (about 20 mg/kg, N=5 epochs for each animal). Data are presented as mean #s.e.m.

FIG. 7M shows electrical stimulation of cell bodies in the VTA produced robust DA transients in the NAc, though lower in amplitude compared to NAc stimulation. Stimulation epochs were recorded before and after cocaine administration (20 mg/kg, IP, N=5 epochs for each animal). Data are presented as mean±s.e.m.

FIG. 7N shows a graph illustrating that following administration of cocaine, stimulation-evoked dLight1.1 transients exhibited a significantly slower decay compared to the pre-cocaine transients due to inhibition of DA reuptake.

FIG. 7O shows a bar graph representation of the line graph shown in FIG. 7N.

FIG. 8 shows a POLI fiber interfacing with a multifunctional backend.

FIG. 9A shows representative beam profile images of PMMA-PC waveguide output light distribution at increasing distances (delta) between the fiber tip and the camera sensor.

FIG. 9B shows fitted Gaussian beam profiles for PMMA-PC waveguide at each delta.

FIG. 9C shows numerical aperture (NA) quantification of different polymer waveguides (N=3 fibers) based on beam dispersion method and comparison to values calculated from reported refractive indices (THVP-PMMA, PMMA-PC, PMMA-COC) or reported by manufacturer (silica). PMMA: poly(methyl methacrylate), PC: polycarbonate, COC: cyclic-olefin-copolymer, THVP: terpolymer of tetrafluoroethylene, hexafluoropropylene, and vinylidene fluoride, SEBS: styrene-ethylene-butylene-styrene.

FIG. 10 shows the stability of POLI fiber-integrated CNT electrodes for electrical stimulation. Mean impedance magnitude at the 1 kHz reference frequency of 20 μm CNT electrodes subjected to 100,000 cycles of stimulation pulsing with the following parameters: 200 μA, 50 Hz, biphasic, cathodic-first, 200 us phases with 25 us interphase interval (N=5 electrodes). A slight decrease in impedance may be observed after the first 2000 cycles of stimulation due to the expected electrode conditioning effect, beyond which the impedance is stable.

FIG. 11A shows a schematic of the implantation site. Eight-weeks old Thy1-GCaMP6s mice were implanted with a 400 μm silica waveguide, 400 μm PMMA/THVP waveguide, or 200 μm PMMA/THVP waveguide. All implants targeted the contralateral whisker sensory cortices (S1BF) area.

FIG. 11B shows a schematic of whiskers contralateral to the implantation site being mechanically stimulated with a brush, while neuronal activity was recorded via GCaMP6s photometry.

FIG. 11C shows the average GCaMP6s signal recorded via a 400 μm silica waveguide during the whisker stimulation experiment. Each tick (top) corresponds to a whisker brush. Data is represented as mean±s.e.m.

FIG. 11D shows the average GCaMP6s signal recorded via a 400 μm silica waveguide represented as a stimulation onset aligned response. Data is represented as mean±s.e.m.

FIG. 11E shows the average GCaMP6s signal recorded via a 400 μm PMMA/THVP waveguide during the whisker stimulation experiment. Each tick (top) corresponds to a whisker brush. Data is represented as mean±s.e.m.

FIG. 11F shows the average GCaMP6s signal recorded via a 400 μm PMMA/THVP waveguide represented as a stimulation onset aligned response. Data is represented as mean±s.e.m.

FIG. 11G shows the average GCaMP6s signal recorded via a 200 μm PMMA/THVP waveguide during the whisker stimulation experiment. Each tick (top) corresponds to a whisker brush. Data is represented as mean±s.e.m.

FIG. 11H shows the average GCaMP6s signal recorded via a 200 μm PMMA/THVP waveguide for 20 seconds represented as a stimulation onset aligned response. Data is represented as mean±s.e.m.

FIG. 12A shows a raw electrophysiological recording also shown in FIG. 4B.

FIG. 12B shows the inter-spike interval (ISI) for the electrophysiological data in FIG. 12A quantified throughout periods of tonic and phasic electrophysiological activity.

FIG. 12C shows an ISI threshold of 0.075 seconds (13 Hz) was chosen based on visual assessment to differentiate phasic and tonic activity FIG. 12B.

FIG. 12D shows a graph showing that phasic activity below the threshold of 0.075 seconds (13 Hz) was found to have an average ISI of 0.040 seconds (25 Hz) and tonic activity above the threshold of 0.075 seconds (13 Hz) was found to have an average ISI of 0.13 seconds (7.5 Hz).

FIG. 13A shows photometry recordings in the VTA following implantation with two POLI fibers targeting the NAc and the VTA.

FIG. 13B shows photometry recordings in the NAc (bottom panel) following implantation with two POLI fibers targeting the NAc and the VTA.

FIG. 14A shows a schematic of the core and cladding of a polymer optical waveguide for use within a POLI fiber probe.

FIG. 14B shows a schematic of the total internal reflection at the interface between the core and cladding of an optical waveguide for use with a POLI fiber probe.

FIG. 15A shows a graph of optical power loss for a polycarbonate (PC)/PMMA polymer fiber for use within a POLI fiber probe.

FIG. 15B shows a graph of optical power loss for a polycarbonate (PC)/PMMA polymer fiber for use within a POLI fiber probe.

FIG. 15C shows a graph of optical power loss for a PMMA/THVP polymer fiber for use within a POLI fiber probe.

FIG. 16A shows an illustration of another example POLI fiber or a notched fiber.

FIG. 16B shows a schematic of the notched fiber of FIG. 16A.

FIG. 16C shows images of the notched fiber of FIG. 16A

### DETAILED DESCRIPTION

The present disclosure describes a fiber-based neural interface (e.g., a fiber probe) that may bidirectionally communicate with the brain, spinal cord, muscles, nerves, and other peripheral organ systems across six modalities. The interface may be capable of monitoring the activity of the biological environment through electrical recording (e.g., electrophysiology), optical recording of fluorescent markers (e.g., fiber photometry), chemical recording of electroactive neurotransmitters (e.g., fast-scan cyclic voltammetry (FSCV)), as well as modulating the biological activity through electrical stimulation (e.g., deep brain stimulation), optical stimulation (e.g., optogenetics), and chemical stimulation (e.g., chemical and/or drug delivery and/or gene delivery through a microfluidic channel). This hexa-functional bidirectional probe may also be MRI compatible, enabling its use for correlating local recording or manipulations of circuit function to brain-wide mapping of neural states. The fiber probes disclosed herein, also called polymer-based optical-electrical-chemical neuroLogical interface (POLI) fibers or POLI fiber probes, may also be flexible and biocompatible, enabling their use for chronic long-term studies (e.g., greater than 6 months).

This technology may record and modulate the activity of neurons, muscles, nerve bundles, glia, and/or other organ systems, and it may be used as an investigational research tool in the study of neuroscience, neuromuscular physiology and disorders, as well as a clinical tool for diagnostics, monitoring, and therapeutic interventions.

The fiber probes disclosed herein may be manufactured using convergence thermal drawing. The fiber probes disclosed herein may be MRI-compatible and may be capable of bidirectional optical, electrical, and/or chemical interrogation of neuronal brain circuits in vivo.

The fiber probes disclosed herein may include an optical waveguide that has lower losses and/or higher numerical apertures (NA) than other polymer waveguides, which may permit photometric recording of calcium and dopamine indicator fluorescence, for example, with a signal-to-noise (SNR) that is comparable to commercial silica fibers.

The fiber probes disclosed herein may also include one or more electrodes. The electrodes may have a low impedance, high charge injection capacity (CIC), and/or high cathodic charge storage (CSC), which may permit their application to electrophysiology, electrical stimulation, and/or FSCV-detection of dopamine (DA), for example, in vivo.

Furthermore, the fiber probes disclosed herein may enable low-artifact functional MRI recordings allowing for monitoring of whole-brain effects of deep brain stimulation (DBS). the fiber probes disclosed herein may be flexible and have a small footprint, allowing the fiber probes to be safely implanted into two brain regions, for example, to permit the interrogation of ventral tegmental area (VTA) and/or nucleus accumbens (NAc) DA circuits via electrophysiological and photometric approaches. In another example, the recordings obtained from the fiber probes disclosed herein may reveal changes in DA release and reuptake dynamics in the presence of cocaine, a known DA reuptake inhibitor, illustrating the potential of the fiber probes disclosed herein as a versatile platform for multi-site interrogations of neural circuits. The fiber probes disclosed herein may combine electrophysiological, DBS, optogenetics, fluid delivery, photometry, and/or FSCV capabilities and may be used to advance both fundamental and preclinical neuroscience studies.

### POLI Fiber Probe Design and Fabrication

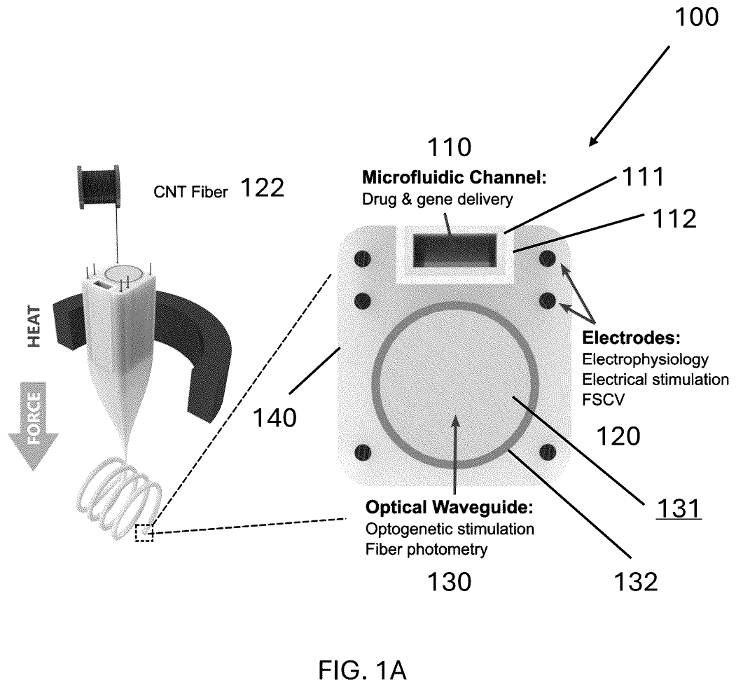

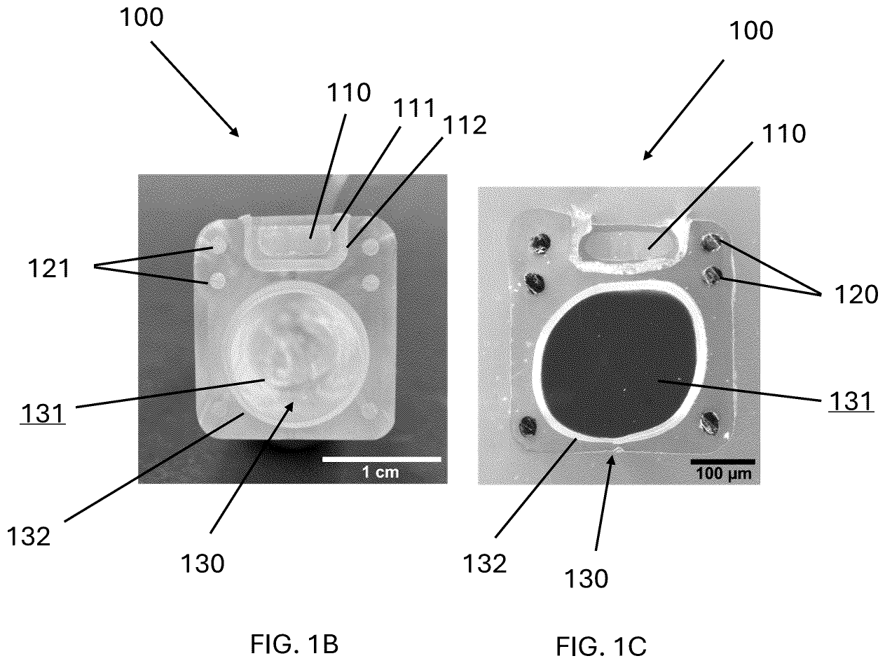

FIGS. 1A-1C show images of a POLI fiber probe 100. The POLI fiber probe 100 may be capable of bidirectional optical, electrical, and chemical interfacing, and can be made using a thermal fiber drawing process, during which a preform produced at the macroscale is heated and stretched into a kilometers-long fiber with microscopic features that may conserve the cross-sectional geometry of the preform as shown in FIGS. 1A-1C. The POLI fiber probe 100 may include a microfluidic channel 110 to enable drug and/or gene delivery, one or more electrodes 120 (e.g., carbon nanotube (CNT) or tungsten (W) microwires), and an optical waveguide 130 for optogenetic stimulation and/or fiber photometry. The POLI fiber probe 100 may also include a housing 140 surrounding the one or more electrodes 120 and the optical waveguide 130. The housing 140 may be made out of an insulating material. For example, the housing 140 may be made out of polycarbonate (PC). Instead of, or in addition to PC, the housing 140 may be made out of any suitable thermoplastic, including but not limited to, Teflon-amorphous fluoropolymer (Teflon-AF), Teflon-perfluoroalkoxy (Teflon-PFA), Cytop (polyperfluoro-butenylvinylether), Hyflon-AD, transparent polypropylene (PP), low density polyethylene (LDPE), high density polyethylene (HDPE), polycarbonate (PC), PMMA, THVP, cyclic olefin copolymer (COC), PMMA, THVP, polyvinyl chloride (PVC), clear PVC, styrene methyl methacrylate (SMMA), polyethylene terephthalate (PET), polyethylene terephthalate (PETG), Ionomer Resin, methyl methacrylate (MABS or Transparent ABS), styrene ccrylonitrile resin (SAN), polystyrene (General Purpose-GPPS), perfluoroalkoxy alkanes (PFA), Hyflon-PFA, THVP 2030GZ (P(TFE-HFP-VDF) Dyneon Terpolymer), GT-PVDF-3 (transparent PVDF), and/or Fluorinatedethylenepropylene (FEP), or Styrene-Ethylene-Butylene-Styrene (SEBS).

The housing 140 may be about 10 μm thick to about 500 μm thick, including all values in between. For example, the housing 140 may be about 10 μm, about 20 μm, about 30 μm, about 40 μm, about 50 μm, about 60 μm, about 70 μm, about 80 μm, about 90 μm, about 100 μm, about 150 μm, about 200 μm, about 250 μm, about 300 μm, about 350 μm, about 400 μm, about 450 μm, or about 500 μm thick, including all values in between. The housing 140 may be about 250 μm to about 400 μm wide and about 300 μm to about 500 μm long, including all values in between. For example, the housing may be about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, or about 400 μm wide. The housing may be about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm long.

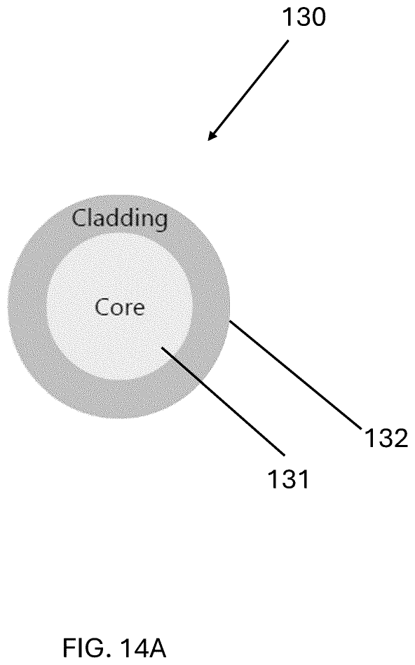

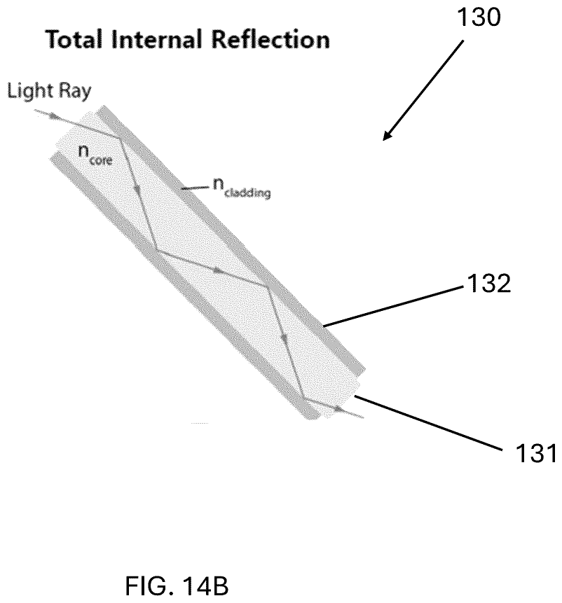

The optical waveguide 130 may include a core 131 and a cladding 132 as shown in FIGS. 1A and 14A. The optical waveguide 130 may be a polymer optical waveguide. This may enable photometric readout fluorescent indicators in vivo and optical stimulation of genetically engineered photo-sensitive proteins (e.g., opsins). The core 131/cladding 132 of the optical waveguide 130 may include, but is not limited to, a combination of poly(methyl-methacrylate) (PMMA)/THVP (polymer of tetrafluoroethylene hexafluoropropylene, vinylidene fluoride). Alternative components of the core 131 and/or cladding 132 of the optical waveguide 130 may include, but are not limited to, Teflon-amorphous fluoropolymer (Teflon-AF), Teflon-perfluoroalkoxy (Teflon-PFA), CYTOP (polyperfluoro-butenylvinylether), Hyflon-AD, transparent polypropylene (PP), low density polyethylene (LDPE), high density polyethylene (HDPE), polycarbonate (PC), PMMA, THVP, cyclic olefin copolymer (COC), PMMA, THVP, polyvinyl chloride (PVC), clear PVC, styrene methyl methacrylate (SMMA), polyethylene terephthalate (PET), polyethylene terephthalate (PETG), Ionomer Resin, methyl methacrylate (MABS or Transparent ABS), styrene ccrylonitrile resin (SAN), polystyrene (General Purpose-GPPS), perfluoroalkoxy alkanes (PFA), Hyflon-PFA, THVP 2030GZ (P(TFE-HFP-VDF) Dyneon Terpolymer), GT-PVDF-3 (transparent PVDF), and/or Fluorinatedethylenepropylene (FEP). For example, the core 131/cladding 132 may include PC/PMMA, COC/PMMA, and/or PMMA/THVP. Preferably, the PMMA forms the core 131 and THVP forms the cladding 132. Preferably, the optical waveguide 130 includes a rigid, flexible, and stretchable polymer.

As shown in FIG. 14B, preferably the refractive index of the core 131 (N_{core}) is greater than the refractive index of the cladding 132 (N_{cladding}). Preferably, the thermomechanical properties (e.g., glass transition temperature and/or melting temperature) of the core 131 and cladding 132 are compatible (e.g., similar). For example, the core 131 may include PMMA and the cladding 132 may include, but is not limited to, THVP, PFA, FEP, CYTOP, Teflon, and/or Hyflon-AD. In another example, the core 131 may include CYTOP and the cladding 132 may include, but is not limited to, Teflon-AF or Hyflon-AD. In yet another example, the core 131 may include COC and the cladding 132 may include, but is not limited to, PMMA, PVC, PP, LDPE, MABS, PFA, THVP, FEP, Teflon, CYTOP, and/or Hyflon-AD

Instead of an optical waveguide 130 including a core 131 and a cladding 132, the optical waveguide 130 may include a gradient index polymer optical fiber.

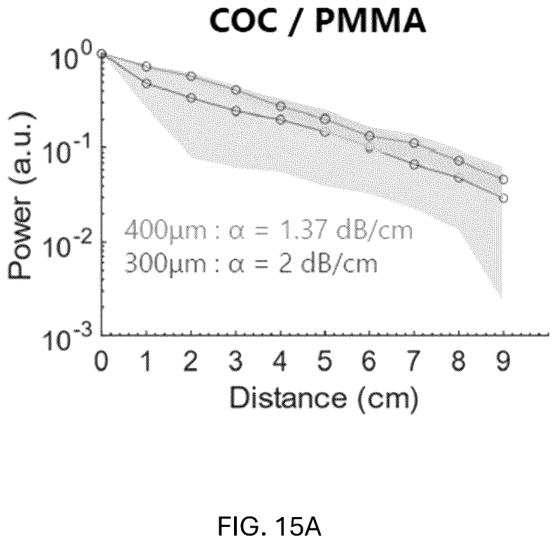

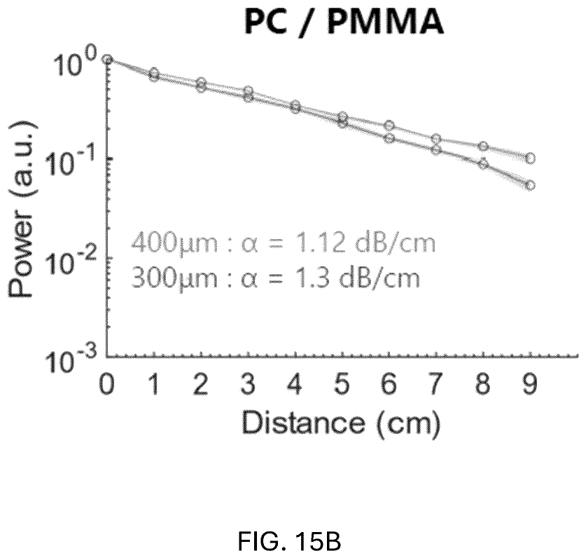

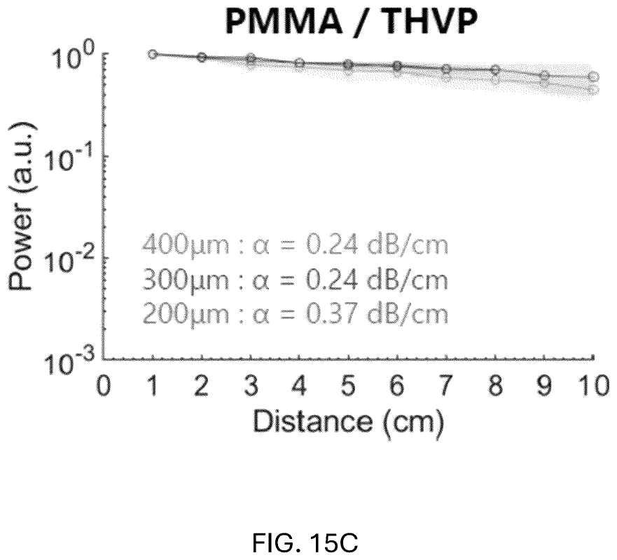

In one embodiment, the optical waveguide 130 may be optically transparent and/or have a high transmittance. In another embodiment, the optical waveguide 130 may include, but is not limited to, a graded index polymer optical fiber, a graded index polymer optical fiber based on amorphous fluoropolymer (Lumiflon), a perfluorinated polymer (CYTOP), and/or a graded index PMMA. These materials were selected for their autofluorescence spectra, refractive indexes, and optical transmission capabilities. In one example, the relative refractive index of the cladding 132 may be less than the relative refractive index of the core 131. For example, PMMA may have a refractive index of 149 and THVP may have a refractive index of 135. This may allow light to bend when traveling through the optical waveguide 130 as shown in FIG. 14B and thus improve the optical transmission capabilities of the optical waveguide 130. FIGS. 15A-15C compare the optical power for three different core 131/cladding 132 combinations of the optical waveguide 130: COC/PMMA (FIG. 15A), PC/PMMA (FIG. 15B), and PMMA/THVP (FIG. 15C). As shown in FIG. 15C, a core 131/cladding 132 combination of PMMA/THVP may have a higher optical power compared to COC/PMMA and/or PC/PMMA.

The POLI fiber probe 100 may also include one or more electrodes 120. The POLI fiber probe 100 may have 1, 2, 3, 4, 5, 6, 7, 8, 9, 10, 11, 12, 13, 14, 15, 16, 17, 18, 19, 20, 21, 22, 23, 24, 25, 26, 27, 27, 28, 29, 30, 31, and/or 32 electrodes. For example, as shown in FIG. 1A, the POLI fiber probe 100 may have 6 electrodes. To enable electrical recording and stimulation of biological activity, a convergence process may be leveraged to incorporate at least one electrode 120 into the POLI fiber probe 100. The electrodes 120 may be about 5 μm to about 100 μm in diameter, including all values in between. For example, electrodes 120 may be about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, or about 100 μm in diameter. For example, the electrodes 120 may be about 10 μm to about 30 μm in diameter, or about 20 μm in diameter.

The electrode(s) 120 may be made of a conducting microwire. For example, the electrode(s) 120 may be made of carbon nanotube (CNT) and/or tungsten (W) microwires. CNT electrodes may be formed from a CNT-doped yarn. The CNT electrodes may enable high signal-to-noise (SNR) ratio electrophysiology recording and high charge injection capacity for delivering stimulation, ensuring long-term, stable bidirectional electrical interfacing. The CNT electrodes may enable small wire stimulation. Additionally, the CNT electrodes may be chemically inert. Instead of or in addition to CNT or tungsten microwires, the electrodes may include one or more conductive elements, including but not limited to, metal (e.g., platinum, platinum-iridium, gold, copper, stainless steel, silver), a coated metal (e.g., iridium oxide coated gold wire or iridium oxide coated titanium) organic conductive material (e.g., carbon nanotubes, graphene, Mxenes, and/or PEDOT: PSS), and/or conductive composite (e.g., a carbon-doped polymer such as carbon-doped polyethylene).

During the convergence process, a microwire of a material (e.g., CNT fiber) with a melting temperature Tₘ (or glass transition temperature T_{g}) significantly higher than the drawing temperature may be fed into a hollow channel (e.g., channel 121 in FIG. 1B) within the preform, which collapses and converges the wire (e.g., electrode 120) into the housing 140 of the resulting POLI fiber probe 100. This approach may enable incorporation of high conductivity metallic electrodes 120 independent of their Tₘ and thereby widens the palette of functional materials that can be integrated into the POLI fiber probe 100. In one embodiment, the electrode 120 may include a 20 μm-diameter carbon nanotube (CNT) fiber or a tungsten (W) microwire into the POLI fiber probe 100, which forms electrodes 120 where they are exposed at the POLI fiber probe tip or along the length of the POLI fiber probe 100. The electrode(s) 120 may be insulated. For example, the electrodes 120 may include an insulation layer. The insulation layer may include any of the materials described above with respect to the housing 140. For example, the electrodes 120 may be insulated with a polymer, including but not limited to, PFA, TPU, TPE, PVC, Polyimide, and/or Parylene-C. The insulation layer may encompass the electrodes 120 and may be removable to expose the electrodes 120. Alternatively, the electrodes 120 may be insulated by the housing 140, which may then be removed to expose the electrodes 120.

To enable chemical recording and stimulation, the unique electrochemical properties of the carbon nanotube electrodes may be leveraged to utilize them as electrochemical sensors of electroactive neurotransmitters through fast-scan cyclic voltammetry (FSCV). The POLI fiber probe 100 may have a high charge injection capacity (CIC) and a low impedance for electrical stimulation and electrophysiological recording. The POLI fiber probe 100 may have a CIC of about 5 mC/c² to about 5000 mC/c². The POLI fiber probe 100 may have an impedance value of about 1 kOhm to about 500 kOhm. Additionally, the high surface area of the CNT electrodes may facilitate the adhesion of DA and increase the sensitivity of the FSCV measurements.

As shown in FIG. 1A, The POLI fiber probe 100 may also incorporate a microfluidic channel 110 capable of delivering chemical, pharmacologic, and/or genetic payloads deep into the tissue of a mammalian subject. The microfluidic channel 110 may include a wall 111 and an elastomeric layer 112. The wall 111 may be made out of any of the materials described above with respect to the housing 140. For example, the wall 111 may be made out of polycarbonate or another suitable material. The wall 111 may be about 5 μm to about 50 μm thick. For example, the wall 111 may be about 5 μm, about 10 μm, about 15 μm, about 20 μm, about 25 μm, about 30 μm, about 35 μm, about 40 μm, about 45 μm, or about 50 μm thick, including all values in between. The elastomeric layer 112 may be made out of any of the materials described above with respect to the housing 140. For example, the elastomeric layer 112 may be made out of styrene-ethylene-butylene-styrene (SEBS), an elastomeric cyclic olefic copolymer, or another suitable elastomer that may be process with the other materials of the POLI fiber probe 100. The elastomeric layer 112 may be about 1 μm to about 50 μm thick. For example, elastomeric layer 112 may be about 1 μm, about 2 μm, about 3 μm, about 4 μm, about 5 μm, about 10 μm, about 15 μm, about 20 μm, about 25 μm, about 30 μm, about 35 μm, about 40 μm, about 45 μm, or about 50 μm thick, including all values in between. The wall 111 and elastomeric layer 112 may separate the microfluidic channel 110 from the housing 140 as shown in FIG. 1A. The elastomeric layer 112 may be mechanically attached to the microfluidic channel 110. The elastomer layer may be sandwiched (e.g., positioned) between the wall 111 and the housing 140 along the whole length of the POLI fiber probe 100. The microfluidic channel 110 may have cross-sectional dimensions of about 25 μm×25 μm to about 300 μm×300 μm, including all values in between. For example, the microfluidic channel 110 may have a cross-sectional dimension of about 25 μm×100 μm to about 40 μm×300 μm. For example, the microfluidic channel 110 may have cross-sectional dimensions of about 20 μm×80 μm to about 60 μm×120 μm. The microfluidic channel 110 may be any suitable shape, including, but not limited to, rectangular, square, circular, or oval. For example, the microfluidic channel 110 may be rectangular in shape to maximize the cross-sectional area of the microfluidic channel 110 given the geometry of the POLI fiber probe 100.

The POLI fiber probe 100 may be made from a macroscale preform using thermal drawing as shown in FIG. 1A. The thermal drawing may be performed at a temperature of about 260-280° C. The macroscale preform may have a cross section of about 5 mm×5 mm to about 10 cm×11 cm, including all values in between. For example, the macroscale preform may have a cross section of about 50 mm×50 mm to about 9 cm×10 cm, about 1 cm×3 cm to about 2 cm×2.5 cm, or for example, about 1.99 cm×2.20 cm. The macroscale preform may be about 10 cm long to about 50 cm long, for example, about 15 cm long to about 40 cm long, about 20 cm long to about 30 cm long, for example about 23 cm long. The macroscale preform may include the components of the housing 140, the wall 111, the elastomeric layer 112, and the core 131/cladding 132 of the optical waveguide 130. As shown in FIG. 1B, channels 121 may be drilled into the housing 140 to form the electrodes 120. The electrodes 120 may be made from a microwire of a material (e.g., CNT fiber) that may be converged from spools into the channels 121 of the housing 140 as described above.

The resulting POLI fiber probe 100 may be about 150 μm wide by about 750 μm long. For example, the POLI fiber probe 100 may about 200 μm×700 μm, about 250 μm×650 μm, about 280 μm×600 μm, about 290 μm×550 μm, about 300 μm×500 μm, about 310 μm×450 μm, about 320 μm×400 μm. For example, the POLI fiber probe 100 may be about 306±17 μm wide by about 342±8 μm long. The POLI fiber probe 100 may be any suitable shape, including, but not limited to, rectangular, square, circular, or oval.

The POLI fiber probe 100 may be operably connected to a printed circuit board (not shown), fluidic tubing (not shown), and/or optical ferrules in a 3D-printed housing (not shown) to form a device that can interface with electrical recording and stimulation equipment, fiber-coupled light sources, and/or micropumps.

### POLI Fiber Probe Connectorization

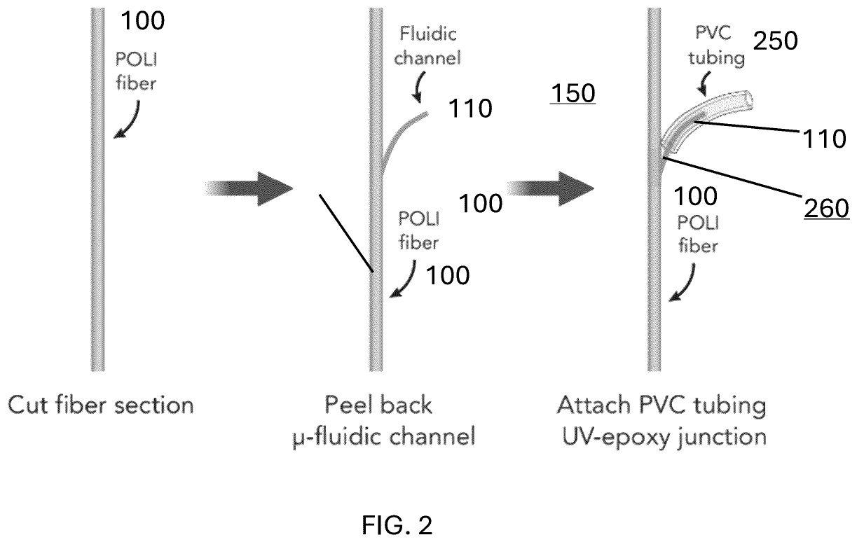

As described above an elastomeric layer 112 may be sandwiched between the microfluidic channel 110 or conduit and the bulk of the fiber probe 100 (e.g., the housing 140), making the microfluidic channel 110 “peelable.” As shown in FIG. 2, a peelable micro-fluidic (μfluidic) channel 110 may be separated (e.g., peeled away) from the bulk of the fiber probe 100. As described above, the μfluidic channel 110 may include a wall 111 (e.g., a PC inner cladding) and an elastomer layer 112. The elastomer layer 112 may surround the wall 111. Preferably, the elastomer layer 112 may have a weak adhesion to the wall 111, enabling the mechanical separation of the μfluidic channel 110 and fluidic interfacing at the fiber probe 100 backend following drawing. For example, styrene-ethylene-butylene-styrene (SEBS) has a relatively weak adhesion to PC. Due to the weak adhesion of the elastomer layer 112 to the wall 111, the microfluidic channel 110 may be able to be mechanically separated (e.g., peeled away) from the bulk of the fiber probe 100. The elastomer layer 112 may be peeled away from the wall 111, enabling the microfluidic channel 110 to be separated from the housing 140.

A μfluidic channel preform may be produced by milling sheets of the wall 111 (e.g., polycarbonate sheets) and consolidating them into about a 3.55×8.52 mm rectangle in a hot press using an aluminum mold and teflon spacer placed in the lumen (185° C., 5 psi, 30 minutes). This rectangle may then be hot-pressed into the elastomer layer (e.g., a layer of SEBS) in an aluminum mold to an outer dimension of about 4.97×9.94 mm (130° C., 5 psi, 10 minutes). The wall 111/elastomer layer 112 (e.g., PC/SEBS) microfluidic preform may then be placed into the rectangular channel on the side of the main housing 140 preform (e.g., a PC preform) and consolidated in the hot press (130° C., 5 psi, 10 minutes). The corners of the preform may be rounded with a 3.55 mm rounded corner end mill to complete the multifunctional fiber preform.

The peeled μfluidic channel 110 may then be inserted into the lumen of a larger-diameter flexible tubing 250 (e.g., a PCV tube) and epoxied in place. The epoxy 260 may be any suitable epoxy, including but not limited to, a two-part epoxy, a UV-curable epoxy, and/or medical grade epoxy. Thus, the μfluidic channel 110 and flexible tubing 250 may be capable of being connected to external tubing and pump via an end-to-end connection. This method may solve a long-standing problem of unreliable connectorization of microfluidic channels.

To connectorize the optical waveguide 130 and electrodes 120, the housing 140 (e.g., polycarbonate cladding) surrounding these components may be chemically etched using dichloromethane, to expose and separate the optical waveguide 130 and the electrodes 120 (e.g., the CNT (or W) microwires), which can then be separately connected to an optical fiber ferrule and standard electrical pin connectors or printed circuit boards using standard techniques.

The POLI fiber probe 100 may also include a custom-made electrical interface board (EIB) (not shown). The probe may also include a 3D-printed casing, which may adjust the implantation depth post-implantation through the use of a vertical carriage, enabling depth-specific recording.

Sections of the POLI fiber 100 may be assembled into a custom dual-fiber probe device (not shown). Then, each electrode 120 (e.g., a CNT microwire) from each of the two POLI fiber probes may be fed through a plated through-hole VIA in the printed circuit board and connected with an electrode interface board (EIB) gold connector pin (e.g., an Open Ephys OEPS-7010). Each of the plated through-holes may be routed to a surface-mounted Omnetics electrical connector (e.g., an Omnetics A79042). Finally, a custom-designed 3D-printed casing may be attached to the bottom of the PCB to enclose all of the connections, with the two fiber probes protruding from the bottom. The two POLI fiber probes may be trimmed precisely to the appropriate lengths to reach the target brain regions, with the help of a 3D-printed cutting mold.

Depth-resolved interrogation of neural circuits may be enabled by adapting a custom-screw and shuttle drive mechanism developed previously to a microdrive compatible with the POLI fiber probe 100. The microdrive may be composed of four functional elements: the drive body, the shuttle that carries the POLI fiber probe 100, a ceramic retaining collar, and a custom screw. Vertical translation of the shuttle may be actuated by rotating the screw.

### Biocompatibility of the POLI Fiber Probe

The dimensions and mechanical stiffness of neural probes have been linked to the foreign body response, which, in turn, determines their long-term functional performance. The integration of ever-increasing functions within devices is often at odds with maintaining their biocompatibility, and most multifunctional neural probes that have been reported to date are fabricated on rigid silicon substrates with metallic components. The POLI fiber probes 100 disclosed herein are fabricated entirely from compliant polymers and thin, flexible microwires to form a probe approximately 150-750 μm in diameter with a low bending stiffness and a low Young's modulus. For example, the POLI fiber probe 100 may have a bending stiffness at 1 Hz of about 10-60 N/m, for example about 20-40 N/m. The POLI fiber probe 100 may have a Young's modulus of about 8 GPa to about 20 GPa, for example, about 12.5 GPa.

### POLI Fiber Probe Implantation

To implant the POLI fiber probe 100, a custom-designed holder may be used to hold and align the probe 100 on a stereotaxic frame to ensure accurate targeting of the implant location. The holder may be a 3D printed holder. Alternatively, the holder may be a ferrule holder.

### POLI Fiber Probe In Vivo Validation

The POLI fiber probe 100 was validated in mice and implanted in several nuclei of the brain such as the ventral tegmental area (VTA), nucleus accumbens core (Nacc), medial prefrontal cortex (mPFC), and the Barrel Cortex. An POLI fiber probe 100 can (1) record electrophysiology, including individual neuron spiking activity, (2) deliver electrical stimulation to evoke activity, (3) record optical readouts of neural activity and neurotransmitter concentrations via fiber photometry, (4) deliver optical stimulation to drive light-sensitive ion channels and proteins (optogenetics), (5) record neurotransmitter concentrations with high temporal resolution via fast-scan cyclic voltammetry, and (6) deliver drug and gene payloads into the brain using a microfluidic channel.

The POLI fiber probe 100 disclosed herein provides a platform to study the brain, muscles, nerves, and other organ systems across several modalities and has several different applications.

First, the POLI fiber probe 100 may be used for investigational neuroscience in the study of substance use disorders (SUD). Understanding neurophysiological principles underlying SUDs demands tools capable of delivering and receiving a diversity of neuronal signals. Furthermore, as SUDs are dynamic conditions emerging over extended periods of time, the ability to monitor neural dynamics over time relies on the biocompatibility and reliable performance of the probes for periods ranging from minutes to months. The POLI fiber probes 100 disclosed herein are MRI-compatible and integrate electrical recording and stimulation, optical recording and stimulation, drug and gene delivery, and chemical sensing via FSCV. Thus, the POLI fiber probe 100 may be leveraged for correlating local recording and/or manipulation of neural circuit function to the brain-wide mapping of neural states associated with withdrawal and compulsive drug seeking in substance use disorders, including, for example, opioid use disorder (OUD).

Second, the POLI fiber probe 100 may be used for distributed electrophysiology recording and electrical stimulation in the muscles, to control prosthetic limbs or to prevent muscle atrophy after an injury, nerve damage, and/or stroke.

Third, the POLI fiber probe 100 may be used for recording or stimulation of peripheral nerves to control prosthetic limbs and/or to provide tactile feedback to the user of prosthetic devices.

Fourth, the POLI fiber probe 100 may be used to deliver patterned electrical stimulation along the length of the POLI fiber probe 100 to re-establish peristalsis in the GI tract, for example, to treat incontinence and/or disorders of the GI system.

Fifth, the POLI fiber probe 100 may be used for the diagnosis and treatment of drug-resistant epilepsies. The POLI fiber probe 100 may be used to localize seizure onset zones and ablate pathologic tissue to prevent seizure onset. This may be achieved using the POLI fiber probe 100 for electrophysiology recording in the brain (e.g., as a stereo EEG electrode) while also locally delivering drugs to transiently inhibit brain regions and observing the effects on the seizure onset network. After localizing the seizure onset zone via electrophysiology and drug delivery, targeted ablation may be performed with the POLI fiber probe 100 through coupling a CO₂ laser to the optical waveguide, delivering electrical or radiofrequency (RF) ablation, and/or chemical and/or pharmacological ablation by delivery of drug payloads through the microfluidic channel.

### Multimodal Probing of Neural and Neuromuscular Circuits

Numerous methods have been developed to record and modulate activity in the nervous system across different domains, including electrical, optical, and chemical. Each method may have its own set of advantages and constraints, particularly regarding the spatial and temporal resolution, the sensitivity and selectivity, and whether it requires genetic manipulation. Despite recent advances in technologies for performing different recording and modulation techniques in the brain and nervous system, these different techniques are rarely integrated together into a single device due to their disparate design constraints. Previous devices have at most allowed combining three techniques for recording and modulation into one platform.

In contrast, the POLI fiber probe 100 disclosed herein enables concurrent recording and modulation with six techniques including: (1) electrophysiology, (2) electrical stimulation, (3) optical recording via fiber photometry, (4) optogenetic stimulation, (5) chemical recording of neurotransmitter species via FSCV, and (6) local delivery of drug and gene payloads through a fluidic channel. By combining these techniques into a single probe, the POLI fiber probe 100 disclosed herein allows for the probing of the interplay between electrophysiological and neurochemical signaling during behavior, as well the studying of the neural responses to deep brain stimulation (DBS), a technique that is widely used clinically for treating the motor symptoms of Parkinson's disease as well as a number of other disorders, but whose mechanism of action in the brain remains poorly understood. Furthermore, the POLI fiber probe 100 disclosed herein may enable the local delivery of drugs or genetic payloads while concurrently recording their effect on neural activity. Finally, the POLI fiber probe 100 disclosed herein can function in the MRI environment, allowing the aforementioned local neural circuit recording and modulation techniques to be linked to whole-brain activity during fMRI. Thus, the POLI fiber probe 100 disclosed herein offers advantages over the currently existing technology by combining the broad range of techniques outlined herein.

Compatibility of POLI Fiber Probes with fMRI

Although ubiquitous in rodent models, photometric, electrophysiological, or FSCV recordings are seldom performed in humans due to their invasiveness. In contrast, functional magnetic resonance imaging (fMRI) is a non-invasive technique that is commonly used to evaluate neurophysiological states in a mammalian subject (e.g., a human subject). To correlate local electrophysiological signatures to brain-wide activity maps, and thus translate insights from rodent studies to development of circuit-based interventions, probes that are safe and exhibit minimal “shadow” artifacts in MRI are desired. Although electrophysiological recordings have previously been acquired during fMRI, they present a formidable challenge as the high-Tesla magnets and radio-frequency fields used for fMRI introduce significant noise into electrophysiological measurements. In contrast, photometric recordings of ion and neurotransmitter fluxes may offer an artifact-free, straightforward means to record activity of broad or genetically-restricted groups of neurons during brain-wide fMRI scans in high-Tesla magnets. The POLI fiber probe 100 disclosed herein may enable both electrical stimulation and fiber photometry recording in the fMRI environment, allowing local recording and neural circuit manipulations to be correlated to brain-wide activity. The POLI fiber probe 100 may also have a low magnetic susceptibility (e.g., χₘ of about −10 ppm) making it suitable for use with MRI.

The POLI fiber probes 100 described herein can be utilized for a variety of applications in research and healthcare. For example, they may be leveraged for basic neuroscience research aimed at investigating neural dynamics and exploring the effects of neuromodulation. A POLI fiber probe 100 may also be used as a medical device, with potential applications including but not limited to, distributed electromyography (EMG) recording in muscles for prosthetics interfacing and rehabilitation medicine, stimulating in muscle to prevent muscle atrophy after injury or stroke, local drug delivery into the brain, muscles, or peripheral organs, recording and stimulation in the brain for neurodiagnostics and monitoring, and/or brain computer interfaces.

By providing a long-term, chronic, flexible, multifunctional, bidirectional interfaces across orthogonal modalities the POLI fiber probes 100 disclosed herein may enable novel research studies into neural dynamics and innovative new medical device technologies.

### Manufacturing Scalability of POLI Fiber Probes

The POLI fiber probe 100 may be produced in a scalable thermal drawing process, where macroscopic models (preforms) of the target POLI fiber probe 100 are heated and drawn into hundreds of meters of microstructured fibers with conserved cross-sections geometrically identical to those of the preforms. A 100 meter long fiber may be cut into centimeter sections and outfitted with appropriate back-end connectors, potentially yielding approximately 5000-10000 multifunctional rodent-scale POLI fiber probes. The low-cost, high-throughput fabrication combined with straightforward customization of neural interrogation modalities, may make fiber technology ideally suited for broad dissemination. Furthermore, the components of the POLI fiber probe 100 are drawn from generally biocompatible materials and fabrication techniques.

### An Example POLI Fiber Probe

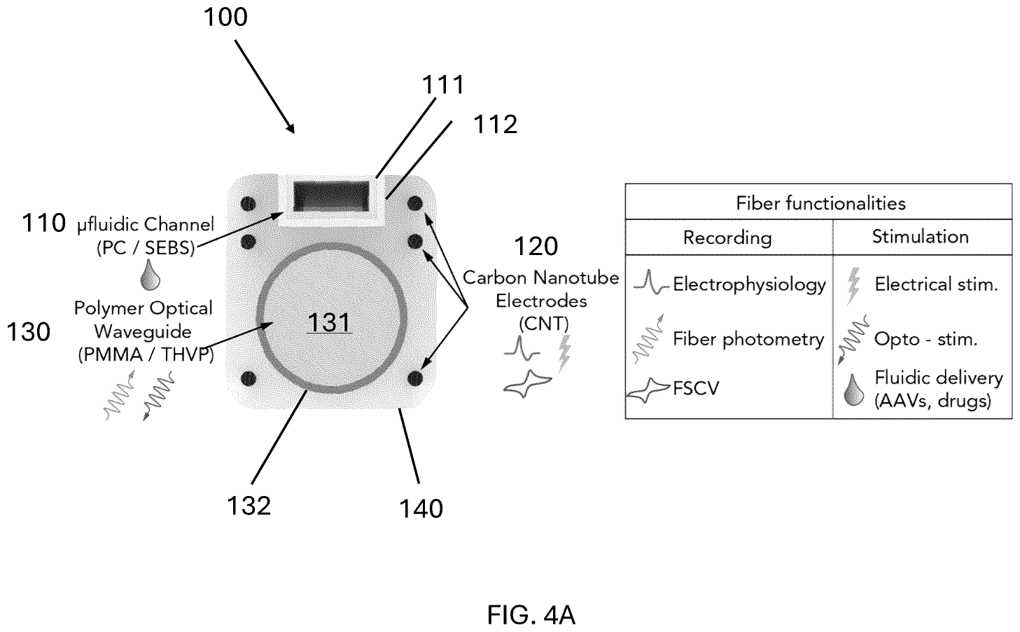

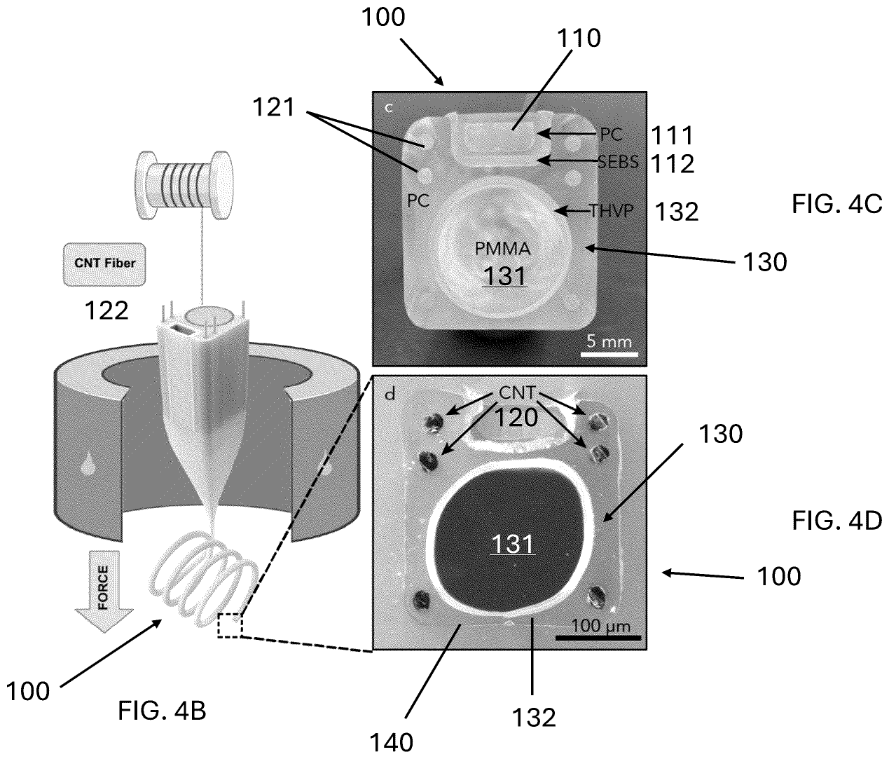

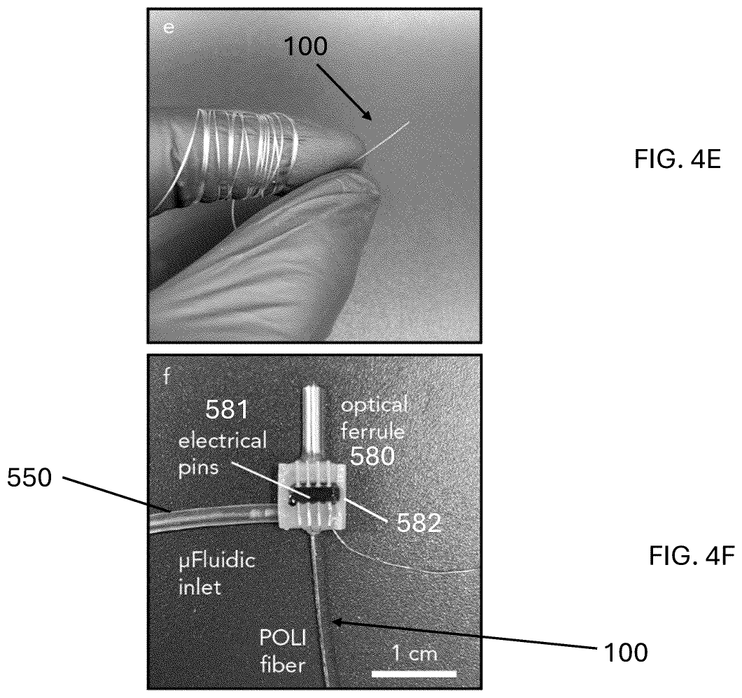

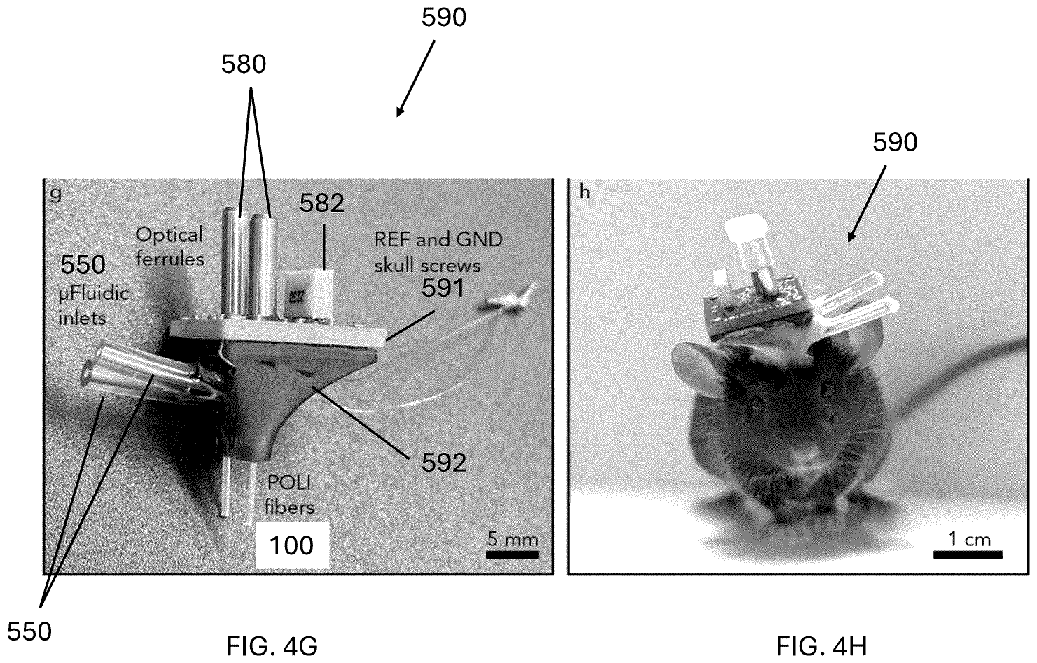

FIG. 4A shows the POLI fiber probe 100. As described above, the POLI fiber probe 100 may include at least one electrode 120, a microfluidic channel 110, an optical waveguide 130, and a housing 140. The optical waveguide 130 may include a core 131 and a cladding 132. The microfluidic channel 110 may include a wall 111 and an elastomer layer 112. As described above, the wall 111 and the elastomer layer may be made out of any of the materials described above with respect to the housing 140. For example, the wall 111 may be made out of polycarbonate or another suitable material. For example, the elastomeric layer 112 may be made out of styrene-ethylene-butylene-styrene (SEBS), an elastomeric cyclic olefic copolymer, or another suitable elastomer that may be process with the other materials of the POLI fiber probe 100. The wall 111 may be about 5 μm to about 50 μm thick, including all values in between. The elastomeric layer 112 may be about 1 μm to about 50 μm thick. The wall 111 and elastomeric layer 112 may separate the microfluidic channel 110 from the housing 140 as shown in FIG. 4A. The elastomeric layer 112 may be mechanically attached to the microfluidic channel 110. The elastomer layer may be sandwiched (e.g., positioned) between the wall 111 and the housing 140 along the whole length of the POLI fiber probe 100.

As shown in FIG. 4A, the POLI fiber probe 100 may include six 20 μm-diameter CNT yarn electrodes. The optical waveguide 130 may be about 200 μm in diameter an may include a PMMA core 131 and a THVP cladding 132. The optical waveguide 130 may be embedded within the housing 140. The housing 140 may be an insulating polycarbonate (PC) cladding.

The microfluidic channel 110 may have lateral dimensions of about 40 μm×100 μm. The wall 111 may be a PC wall may be separated from the housing 140 via a thin elastomer layer 112. The elastomer layer 112 may be a layer of styrene-ethylene-butylene-styrene (SEBS) elastomer (FIG. 4A).

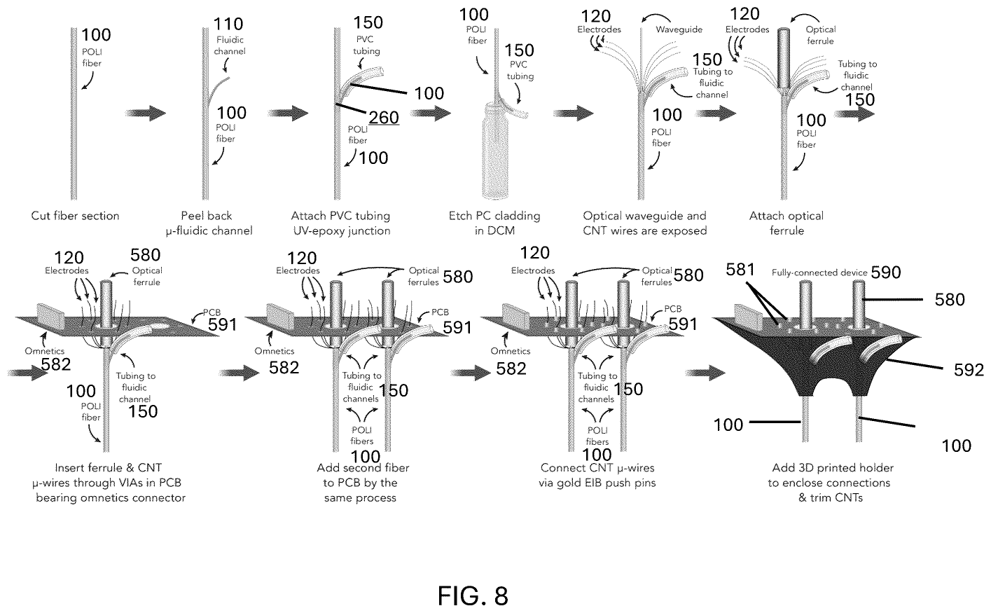

The constituent polymers for the POLI fiber probe 100 may be selected based on the relative similarity of their glass transition temperatures (e.g., about 80-185° C.) to enable thermal co-drawing. Table 1 provides the glass transition temperatures (Tg) and refractive index values of example polymers that may be used in the POLI fiber probe 100. Additionally, PMMA and THVP were selected for the core 131 and cladding 132, respectively, due to their low absorption and high refractive index contrast in the visible range. The thin elastomer layer 112 (e.g., the SEBS layer) surrounding the microfluidic channel 110 may facilitate connections of the microfluidic channel 110 to the fiber probe 100. For example, as described above the elastomer layer 112 allows for the peeling off (e.g., mechanical separation) of the microfluidic channel 110 from the fiber probe 100, which may enable an easier fluidic connection. Additionally, due to its relatively weak adhesion to PC, SEBS may enable peeling of the microfluidic channel 110 from the side of the fiber probe 100, as described above, for in-line fluidic connection to external tubing, and isolating the optical and electrical components for connectorization (FIGS. 2 and 8).

TABLE 1
Glass transition temperature (T_{g}) and refractive index values of polymers that may
be used in a POLI fiber 100. PMMA: poly(methyl methacrylate), PC: polycarbonate, COC: cyclic-
olefin-copolymer, THVP: terpolymer of tetrafluoroethylene, hexafluoropropylene, and vinylidene
fluoride, SEBS: styrene-ethylene-butylene-styrene.

| | Refractive | Glass transition | | Melting |
|---|---|---|---|---|
| | index, | temperature | Transmittance | Temperature |
| Material | n (wavelength) | T_{g} (° C.) | (T) (%) | Tₘ (° C.) |
| PMMA | 1.4956 (488 nm) | 105-120 | 89-92 | |
| PC | 1.5976 (488 nm) | 130-170 | 86-91 | |
| COC | 1.543 (488 nm) | 158 | 91 | |
| THVP | 1.35 (589 nm) | | 94 | 130 |
| SEBS | N/A | 90 | | |
| Teflon-AF | 1.31 | 160-240 | >95% | |
| Cytop | 1.34 | 108 | >95% | |
| (polyperfluoro- | | | | |
| butenylvinylether) | | | | |
| Hyflon-AD | 1.33 | 90-140 | 80 | |
| Transparent | 1.347 | 0 | | |
| Polypropylene | | | | |
| (PP) | | | | |
| LDPE | 1.476 | −110 | 4.4-94 | |
| Clear PVC | 1.381 | 70-100 | Up to 97% | |
| Styrene Methyl | 1.59 | 125 | 89-92.8 | |
| Methacrylate | | | | |
| (SMMA) | | | | |
| PET | 1.575 | 73-78 | 87-92.1 | |
| PETG | 1.55 | 79 | 92 | |
| Ionomer Resin | 1.49 | | 93.4 | |
| MABS | 1.52 | | 86 | |
| (Transparent | | | | |
| ABS) | | | | |
| Styrene | 1.57 | 100 | 86.2-89.3 | |
| Acrylonitrile | | | | |
| Resin (SAN) | | | | |
| Polystyrene | 1.6 | 95 | 88-90 | |
| (General Purpose- | | | | |
| GPPS) | | | | |
| PFA, Teflon-PFA, | 1.35 | 90 | 91-96% | 305 |
| Hyflon-PFA | | | | |
| GT-PVDF-3 | 1.42 | | 92% | 170 |
| (Transparent | | | | |
| PVDF) | | | | |
| FEP | 1.34 | 80 | 94% | 270 |

To fabricate the POLI fiber probe 100, a macroscale preform (e.g., a 5 mm×5 mm to about 10 cm×11 cm cross-section, for example about 1.99 cm×2.20 cm cross-section, about 22.9 cm long) containing the components of the housing 140, core 131, cladding 132, wall 111, and elastomer layer 112 (e.g., PC, PMMA, THVP, and SEBS) may be produced through machining and consolidation techniques. Channels 121 were drilled into the housing 140 (e.g., PC body) to accommodate the electrode microwire (e.g., CNT yarn). The preform was then thermally drawn at a temperature of about 260-280° C. and the 20 μm diameter electrode microwire 122 (e.g., CNT yarn) may be converged from spools into the open channels 121 of the preform to form the electrodes 120 (FIGS. 4B and 4C). The drawing procedure may deliver a flexible POLI fiber probe 100 with cross-sectional dimensions of about 150 μm to about 165 μm to about 750 μm to about 825 μm. For example, the POLI fiber probe 100 may have cross-sectional dimensions of about 250 μm by about 400 μm to about 700 μm to about 800 μm, for example about 306±17 μm by about 342±8 μm (FIGS. 4D and 4E). FIG. 4D shows an optical micrograph of the POLI fiber 100 cross-section highlighting the cross-sectional geometry and embedded electrodes 420. FIG. 4E shows a photograph highlighting the flexibility and microscopic size of the POLI fiber probe 100. Minor distortion of the PMMA/THVP optical waveguide 130 may be observed but may not significantly impact transmission losses of the PMMA core 131 as discussed below.

To facilitate interfacing with electrical recording and stimulation equipment, fiber-coupled light sources, and tubing leading to micropumps, sections of the POLI fiber probe 100 may be combined into an assembly 590 as shown in FIGS. 4G and 4H. Preferably the section of the POLI fiber probe 100 for the an assembly 590 is at least 1 cm long. The assembly 590 may include a printed circuit board (PCB) 591, fluidic tubing 150, and optical ferrules 580 in a 3D-printed housing 592 to permit attachment of either one (FIG. 4E) or two (FIG. 4F) POLI fibers. The 3D-printed housing 592 may be made of any material that may be 3D printed, including, but not limited to, resin or acrylonitrile butadiene styrene (ABS). The PCB 591 may be about 6 mm×6 mm to about 25 mm×25 mm, including all values in between. For example, FIG. 4F shows a fully assembled POLI fiber probe 100 with electrical pins 581, an optical ferrule 580, and tubing 150 that forms microfluidic inlet. While FIG. 4F includes two POLI fiber probes 100, a single POLI fiber probe 100 may also be used. For example, the assembly 590 may include one POLI fiber probe 100, two POLI fiber probes 100, three POLI fiber probes 100, or four POLI fiber probes 100. If the assembly 590 includes more than one POLI fiber probe 100, each POLI fiber probe 100 of the assembly 590 may be implanted into separate brain regions, which may allow for the study of a network and/or connected brain regions. For example, if the assembly 590 includes four POLI fiber probes 100, two POLI fiber probes may be implanted into each brain hemisphere.

FIG. 4G shows a photograph of a dual-implant assembly 590 that leverages PCB and Omnetics connector to facilitate connectorization. FIG. 8 shows the preparation of the assembly 590 from a POLI fiber probe 100 and the steps for connectorization. This assembly 590 may simplify the implantation process of fiber devices targeting the VTA and NAc. The dual-implant assembly 590 may enable the assembly 590 to probe a network in the brain. For example, the dual-implant assembly 590 may be able to probe the projection from one network and/or nucleus to another network and/or nucleus in the brain. The dual-implant assembly 590 may also allow for a decreased implantation time compared to the implantation time required for two separate implants. The dual-implant assembly 590 may also improve accuracy with implantation. For example, dual-implant assembly 590 may have a fixed distance between each POLI fiber probe 100, reducing potential errors with implantation.

The microfluidic channel 110 may be prepared as described above. The microfluidic channel 110 may be mechanically separated (e.g., peeled away) from the bulk of the fiber probe 100 as described above. The peeled microfluidic channel 110 may then be inserted into the lumen of tubing 550 (e.g., a PCV tube) and epoxied in place. The epoxy 260 may be any suitable epoxy, including but not limited to, a two-part epoxy, a UV-curable epoxy, and/or medical grade epoxy. Thus, the peeled microfluidic channel 110 and tubing 550 may be capable of being connected to external tubing and pump via an end-to-end connection.

The electrodes 120 of the POLI fiber probe 100 may be separated from the optical waveguide 130 of the POLI fiber probe 100. The electrodes 120 may be chemically etched and/or mechanically stripped to separate the electrodes 120 from the optical waveguide 130. For example, the cladding 132 may be dissolved (e.g., using chemically etching) or stripped away (e.g., using mechanical stripping) to separate the electrodes 120 from the optical waveguide 130. The optical waveguide 130 of the POLI fiber probe 100 may then be connected to a optical ferrule 580. The electrodes 120 of the POLI fiber probe 100 may be threaded through holes on a corresponding PCB 591 and capped with electrical pins 581. The PCB 591 may bore one or more electrical pins 581 that may be connected to an electrical connector 582 (e.g., a 16-channel Omnetics® electrical connector or another suitable electrical connector) and the openings may allow for one or more (e.g., 1 or 2) optical ferrules 580 to be threaded through the PCB 591. The PCB 591 may also be attached to a housing 592.

In each assembly realization, each individual POLI fiber probe 100 remained capable of performing all six functions. The assembly 590 may weigh about 1 g to about 5 g, for example about 1.5 g to about 3 g, or about 2.292 g. The complete assembly 590 may be compatible with stereotaxic brain surgery in mice (FIG. 4H). For example, FIG. 4H shows a photograph of a freely moving mouse with the dual-fiber implant 590. The assembly 590 may also be used in a mammalian subject (e.g., a human). To adapt the assembly 590 for use in a mammalian subject, the length of the POLI fiber probes 100 may be increased. For example, the length of the POLI fiber probe 100 may be increased by about 1 mm, about 10 mm, about 20 mm, about 30 mm, about 40 mm, about 50 mm, about 60 mm, about 70 mm, about 80 mm, about 90 mm, about 100 mm, about 1 cm, about 2 cm, about 3 cm, about 4 cm, about 5 cm, about 6 cm, about 7 cm, about 8 cm, about 9 cm, or about 10 cm.

### POLI Fiber Characterization

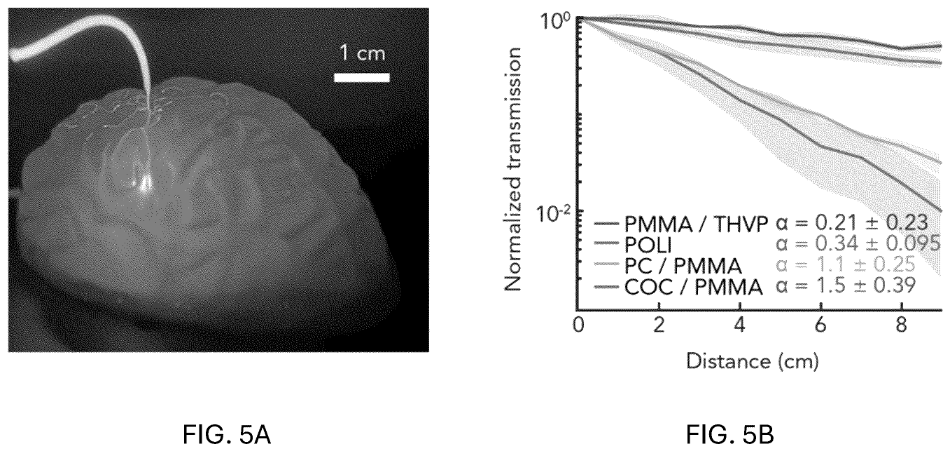

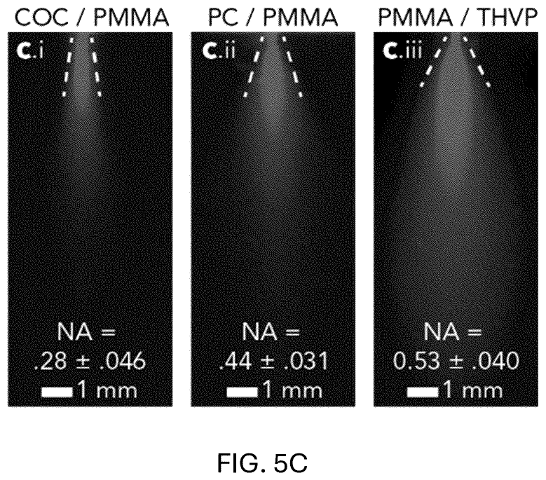

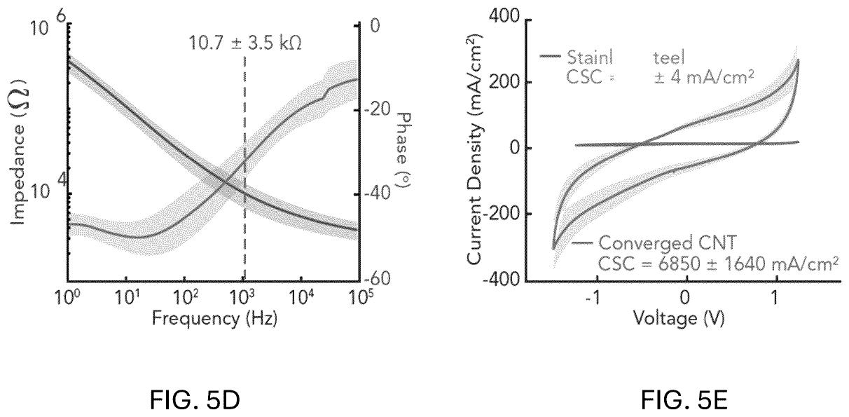

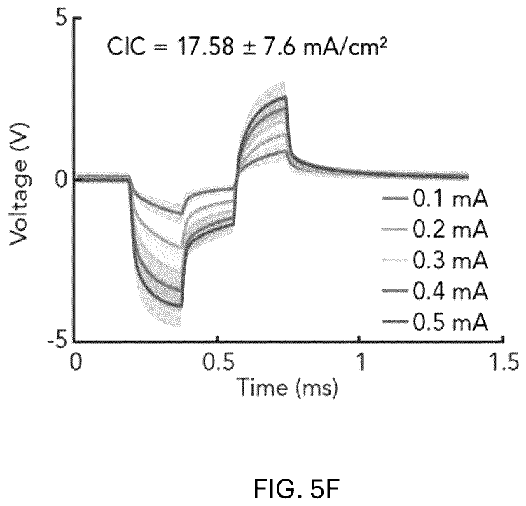

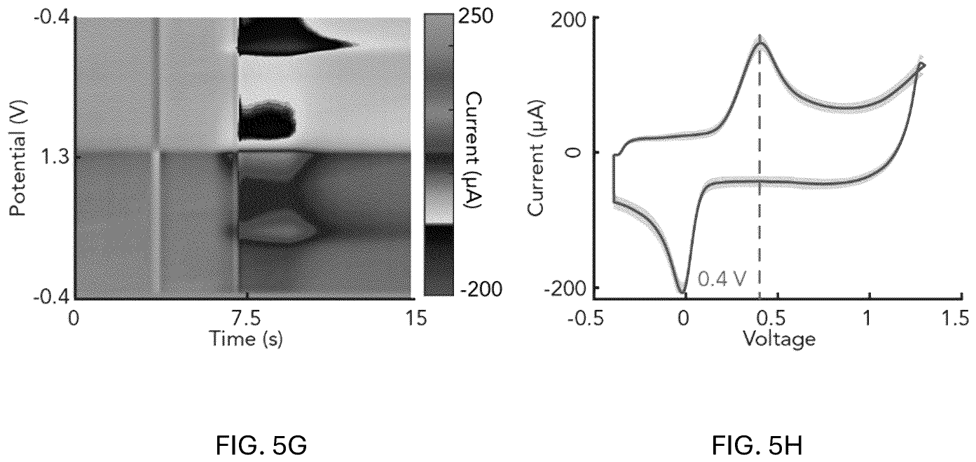

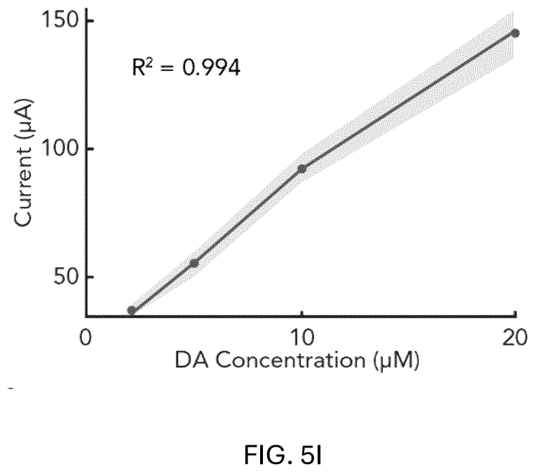

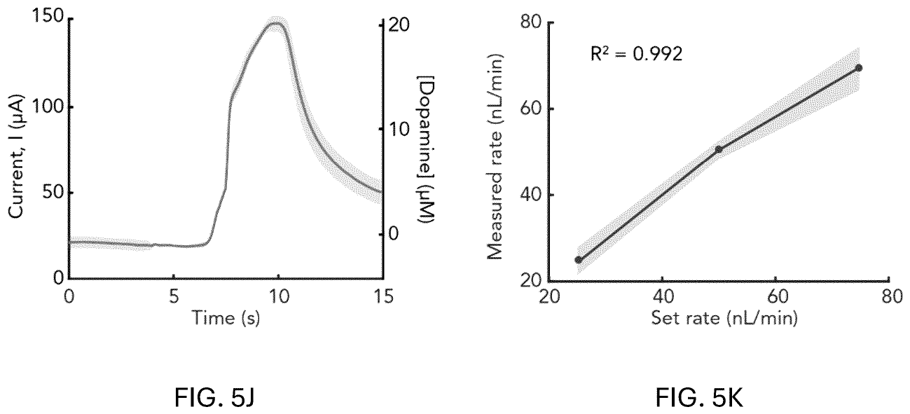

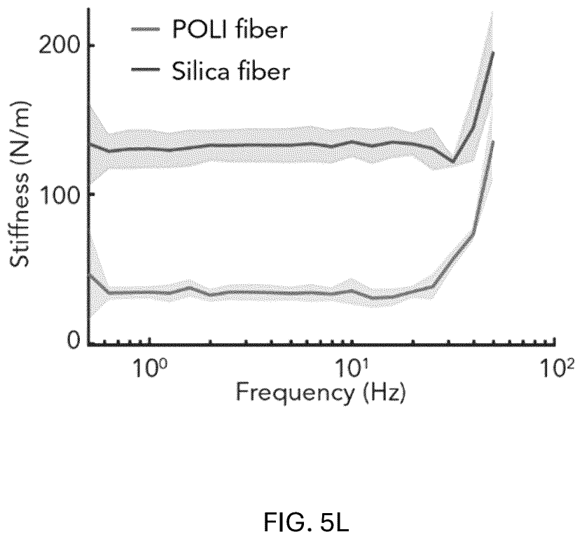

The functional performance of each POLI fiber probe 100 element was first assessed in situ. To quantify the ability of the optical waveguides 130 with PMMA (n_{PMMA}=1.4956, λ=488 nm) core 131 and THVP (n_{THVP}=1.35, λ=589 nm) cladding 132 to collect and transmit optical signals (FIG. 5A), their loss coefficients (a, dB/cm, FIG. 5B) and numerical apertures (NA, FIGS. 5C and 5D) were measured. As shown in FIG. 5A, blue light may be visible along the patch cord due to high input optical power, which may permit visual observation of the fluorescent response. Additionally, the power of optical leakage along the fiber due to impurities or microscale defects may be orders of magnitude below the power of light transmitted to the fiber tip, and as observed, may not elicit a fluorescent response along the length of the fiber probe 100.

Other multifunctional fiber-based probes rely on waveguides with PCores (n_{PC}=1.60, T_{g}=130-170° C.) and cyclic olefin copolymer (COC, n_{COC}=1.55, T_{g}=158° C.) cladding. While suitable for optogenetics, these waveguides may not enable photometric recordings due to the potential for autofluorescence of the polycarbonate core. Thus, the loss coefficients and NA of polymer waveguides composed of several combinations of core/cladding: PMMA/THVP, PC/PMMA, and COC/PMMA with similar core-cladding diameters (400 μm/420 μm) were evaluated (FIGS. 15A-15C). Consistent with the high refractive index contrast between PMMA and THVP and lower absorption of PMMA as compared to PC in the visible range, it was found that the largest NA=0.53±0.04 and lowest loss coefficient α=0.55±0.14 dB/cm (n=3 samples) for PMMA/THVP fibers as compared to COC/PMMA (NA=0.28=0.05, α=1.5±0.39 dB/cm) and PC/PMMA (NA=0.44±0.03, α=1.10±0.25 dB/cm) waveguides, respectively (FIGS. 5B and 5C).

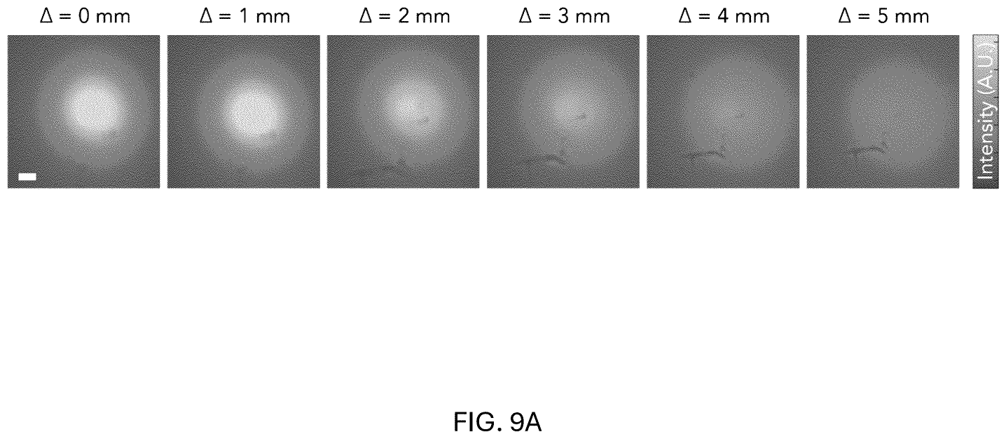

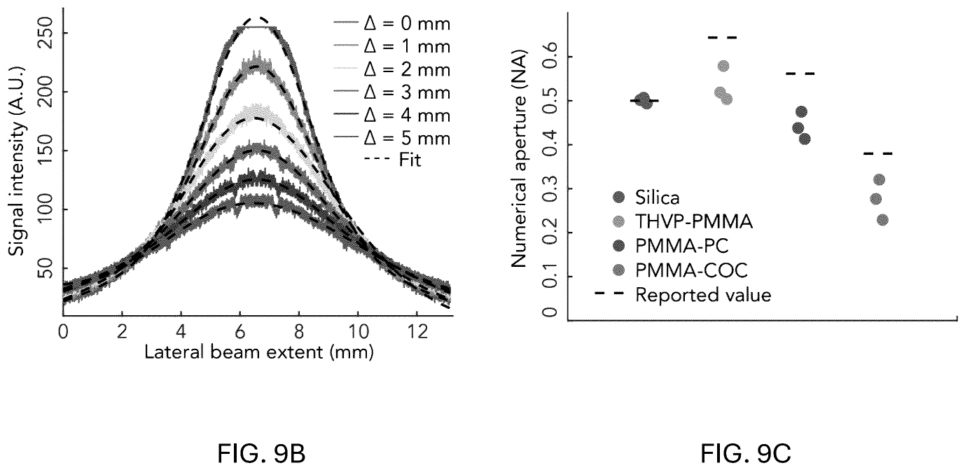

PMMA/THVP fibers may exhibit a higher NA than commercial silica fibers with a similar core diameter (400 μm, manufacturer NA=0.5, measured NA=0.50±0.022), which may yield improved light collection for fiber photometry. Due to minor deviations from the cylindrical shape in custom-drawn fibers, experimentally measured NA values may be slightly lower (e.g., about 17-27%, FIGS. 9A-9C) than those calculated from the reported refractive indices but follow anticipated trends. Although polymer waveguides may have greater loss coefficients than silica fibers, the resulting attenuation may be minimal at rodent-brain scales (e.g., about 6.1% for 5 mm) and the resulting attenuation (e.g., 58.8% for 7 cm, FIG. 5A) may also be likely for studies in larger organisms such as non-human primates and/or mammalian subjects (e.g., humans). The resulting attenuation may be decreased by using a different material for the core 131 and/or cladding. The resulting attenuation may be decreased by using a gradient index polymer optical fiber instead of a optical waveguide 130 with a core 131 and cladding 132. Additionally, the substantially lower stiffness of polymer-based fibers compared to silica fibers may improve mechanical compatibility with brain tissue.

The POLI fiber probe 100 may have a numerical aperture of about 0.2 to about 0.6, for example about 0.21 to about 0.59, about 0.23 to about 0.58, about 0.24 to about 0.57, about 0.25 to about 0.56, about 0.26 to about 0.55, about 0.27 to about 0.53, including all ranges in between. The POLI fiber probe 100 may have a loss coefficient α of about 0.5 to about 2.0, for example about 0.51 to about 1.99, about 0.52 to about 1.98, about 0.53 to about 1.97, about 0.54 to about 1.96, or about 0.55 to about 1.95, including all values in between.

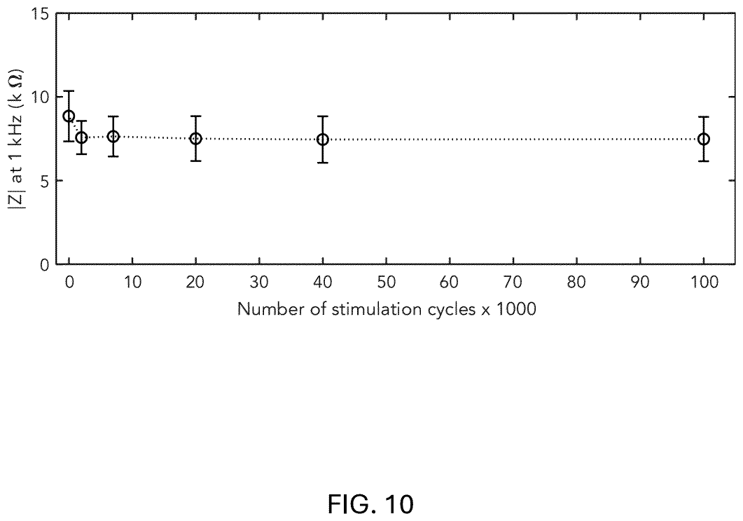

Next, the electrochemical properties of the electrodes 120 (e.g., the 20 μm-diameter CNT electrodes) embedded within the POLI fiber 100 were evaluated. Electrochemical impedance spectroscopy (EIS) revealed characteristic capacitive behavior (FIG. 5D), with a mean |Z| value of about 10.7±3.5 kΩ at 1 kHz (n=3 fibers, 18 electrodes) which was only slightly affected by repeated stimulation pulsing (n=5, 100,000 pulses) with a slight decrease of impedance observed after the first 2000 cycles due to electrode conditioning, beyond which the impedance remained stable. (FIG. 10).

CNT electrodes possess a wide electrochemical water window of about-1.5 V to about +1.2 V; within this window, electrical stimulation pulses can be delivered without inducing hydrolysis. The cathodic charge storage capacity (CSCc) of the CNT electrodes in this window may be about 5000 mC/cm2 to about 9000 mC/cm2, for example about 5,500 mC/cm2 to about 8,500 mC/cm2, or about 6850±1636 mC/cm2, which is significantly greater than same-size stainless steel electrodes characterized over the same voltage range (e.g., 98.0±4.1 mC/cm2) (FIG. 5E). The CSC of CNT electrodes is significantly larger than that of standard clinical stimulation electrodes (platinum, CSC=0.55 mC/cm2; iridium oxide, CSC=4 mC/cm2). Additionally, the CSC value measured here for the CNT electrodes may exceed that of previous reports of CNT-based electrodes, which may be attributed to the large electrochemically active surface area of the rough CNT yarn electrodes that is not accounted for in the CSC calculations based on geometric surface area. The charge injection capacity (CIC) of the CNT electrodes was measured via voltage transient testing with biphasic current pulses, increasing in amplitude until the maximum cathodic excursion potential (E_{mc}) crossed the water window limit of −1.5 V.

The CNT electrodes in the POLI fiber 100 exhibit a CIC of about 5 mC/cm2 to about 30 mC/cm2, for example about 10 mC/cm2 to about 25 mC/cm2, or about 17.6±7.6 mC/cm2, which is substantially higher than that of stainless steel (CIC=1.5±0.5 mC/cm2) as well as clinical alternatives (platinum, CIC=0.35 mC/cm2; iridium oxide, CIC=0.87 mC/cm2), indicating a greater amount of charge that can be safely injected during a stimulation pulse (FIG. 5F). Platinum or platinum-iridium electrodes may also be integrated within thermally drawn polymer fibers via the same convergence process described herein. However CNTs electrodes may have more favorable charge injection characteristics and be compatible with MRIs.

The low impedance and wide electrochemical stability window (e.g., about-1.5 V to about 1.2 V) of CNT electrodes also make them suitable for FCSV. FSCV is commonly performed using brittle carbon fiber electrodes, and CNT yarn offers flexibility, robustness, and greater sensitivity while retaining electrochemical stability. The FSCV capabilities of the electrodes 120 within POLI fibers 100 were first assessed in vitro in a flow cell with solutions of DA in HClO₄ diluted to concentrations of about 2 μM-20 μM in PBS using a standard DA waveform scanning between about-0.4 V to about 1.3 V at a rate of 100 V/s and sampling frequency of 10 Hz (FIG. 5G, 5J, and 5I-5L). A color plot illustrates the current response of the CNT electrodes (e.g., electrodes 120) to 10 μM DA in solution (FIG. 5G), and a corresponding cyclic voltammogram reveals known DA redox peaks (FIG. 5H). The peak current at the oxidation voltage (e.g., about 0.4 V) varies linearly over a range of physiological DA concentrations (FIGS. 5I and 5J, R²=0.994, n=3 fibers), with a limit of detection (LOD) of 0.1916 μM and a limit of quantification (LOQ) of 0.581 μM, suggesting the utility of CNT electrodes for DA concentration measurements in vivo.

The microfluidic channel 110 within POLI fibers 100 (40×100 μm²) were assessed by recording the rate of fluid passage driven by a syringe pump. Consistency (1:1) between the pump and the fiber flow rates was found across the microfluidic channel 110 of multiple POLI fiber probe 100 (FIG. 5K, R²=0.992, n=3 devices).

The stiffness of POLI fibers 100 (about 360-380×400-420 μm², n=3 devices) was assessed via dynamic mechanical analysis (DMA) over a range of frequencies corresponding to heartbeat, respiration, and locomotion (FIG. 5L). Compared to a silica waveguide (diameter=400 μm, n=3 fibers), the POLI fiber 100 exhibited a significantly lower stiffness, which was comparable to previously reported polymer-based multifunctional fibers with long-term tissue stability.

### POLI Fiber Probe 100 Functional Characterization In Vivo

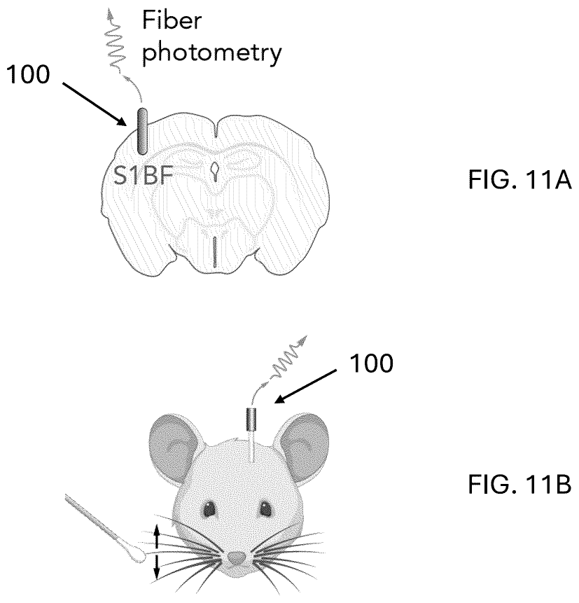

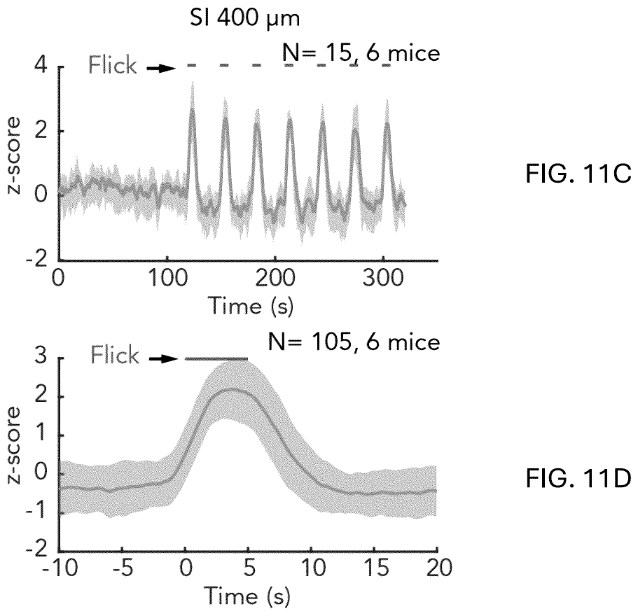

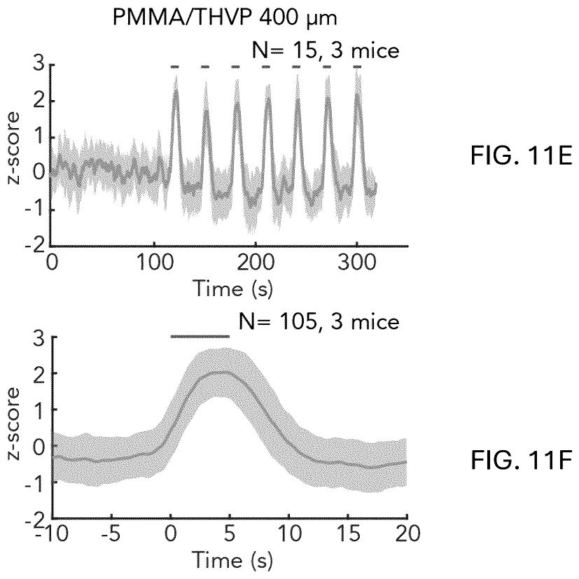

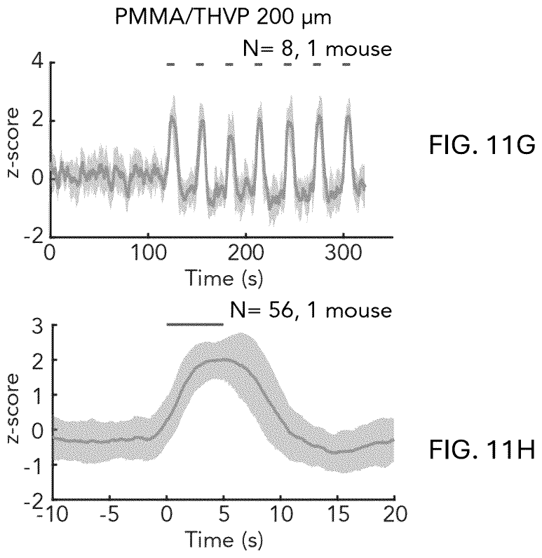

The abilities of POLI fibers 100 to perform photometric recordings of activity indicators, electrical stimulation (including during MRI), and FSCV measurements of DA, were first evaluated. Photometric recordings with PMMA/THVP fiber platform were first compared to those performed with commercial 400 μm diameter silica multimode fibers, the most common photometry platform. PMMA/THVP fibers thermally drawn to about 200 μm or about 400 μm diameter (core/cladding ratio=95%) were implanted into the contralateral whisker sensory cortices (SIBF) of Thy1-GCaMP6s mice broadly expressing a fluorescent calcium indicator GCaMP6s in excitatory neurons (FIG. 11A). The core/cladding ratio refers to the ratio of the core 131 diameter to the cladding 132 diameter. The core/cladding ratio may be about 80% to about 99%, for example about 85% to about 95%, or about 95%. In these mice, 400 μm silica fibers were implanted into the same brain region in the opposite hemisphere (FIG. 11B). Fluorescence increases in response to whisker flicks were recorded by both 200 μm (n=8 trials, 1 female mouse) and 400 μm (n=15 trials, 3 mice—2 male and 1 female) PMMA/THVP fibers with an SNR comparable to that of the 400 μm silica fiber (n=15 trials, 6 mice—3 females and 3 males) (FIGS. 11C-11H).

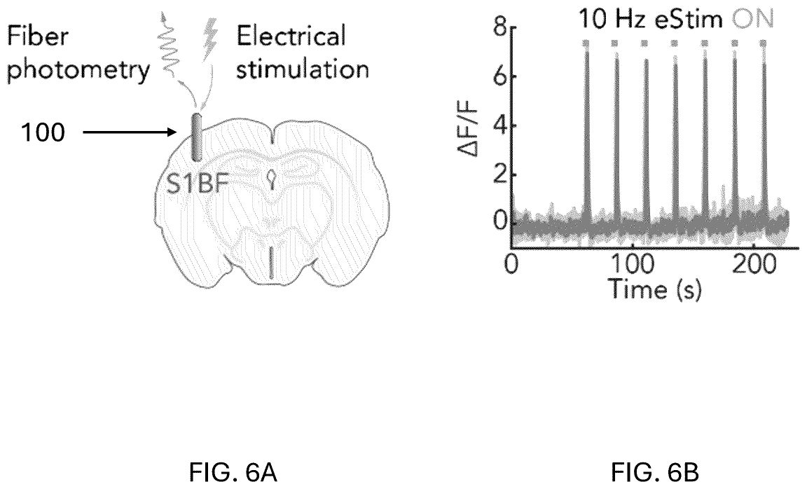

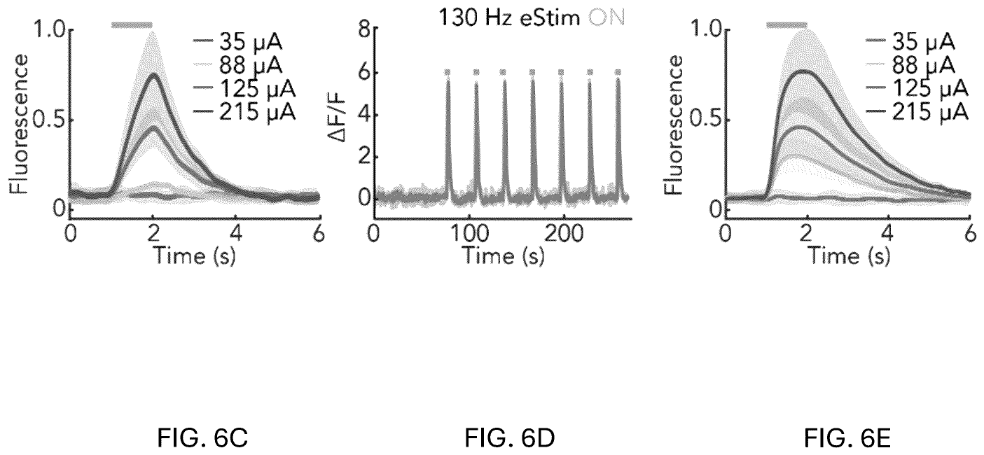

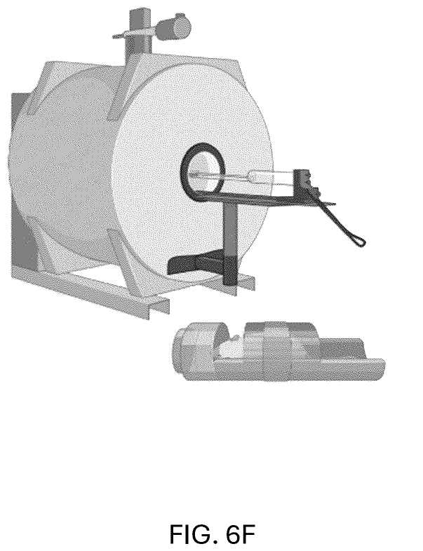

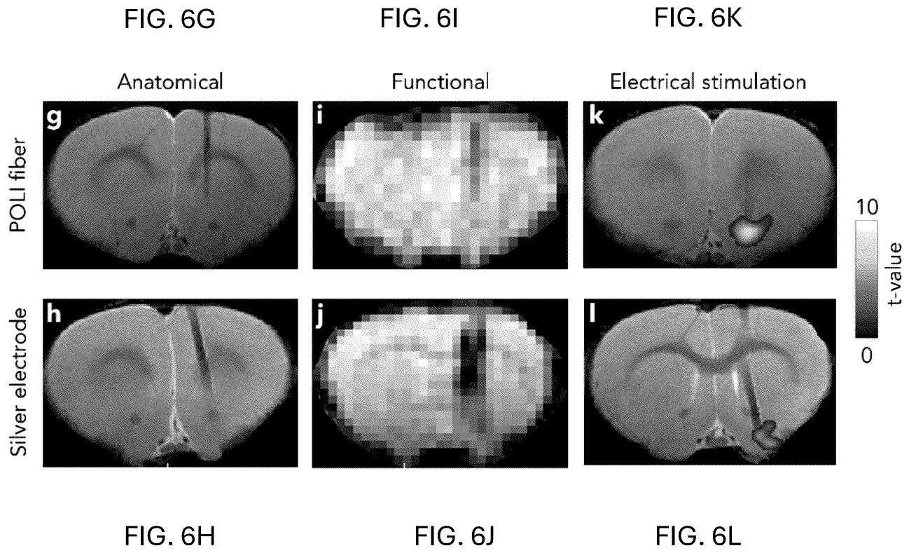

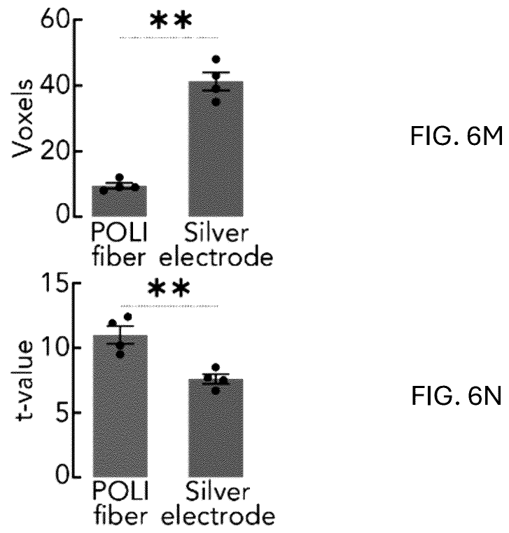

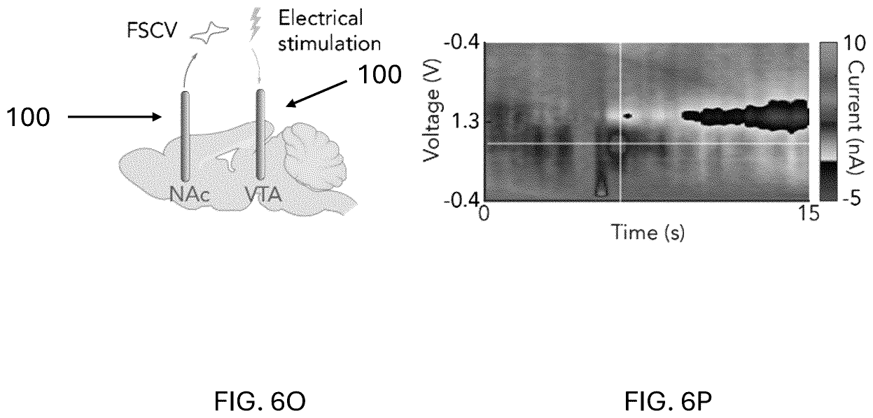

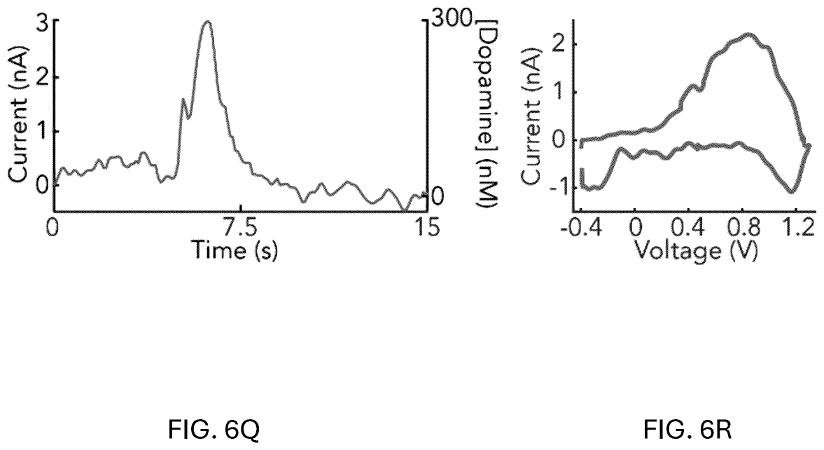

Given that the diameter of the PMMA/THVP waveguides (e.g., the core 131/cladding 132) within POLI fibers is about 200 μm, which may be suitable for photometric recordings, the assembly 590 was then applied to test the ability of the electrodes 120 to drive neural activity and evoke calcium influxes. A POLI fiber probe 100 was implanted in the SIBF of Thy1-GCaMP6s mice, and the embedded CNT electrodes (e.g., electrodes 120) were applied to deliver bipolar electrical stimulation at frequencies of about 10 Hz (FIGS. 6A-6C) and about 130 Hz (FIGS. 6D-6E). These stimulation frequencies and currents varying between about 35-215 μA are typical for therapeutic neuromodulation. Robust increases in normalized GCaMP6s fluorescence (ΔF/F₀) were recorded in response to 10 Hz stimulation above 88 μA, and the threshold was lower for 130 Hz stimulation indicating a dose-dependent response. These observations corroborate the ability of POLI fibers probes 100 to simultaneously deliver electrical stimulation and photometrically record evoked neuronal activity.

The POLI fibers 100 were then applied to deliver electrical stimulation in a preclinical context where changes in blood-oxygen-level-dependent (BOLD) signal recorded during MRI were used to assess the effects of deep brain stimulation (DBS). A POLI fiber probe 100 was implanted in the nucleus accumbens (NAc), a midbrain structure implicated in reward processing, in Sprague Dawley rats (n=8). These experiments were performed in parallel in rats implanted with a pair of silver electrodes (e.g., about 356 μm in diameter) traditionally used for DBS during MRI in rodents (FIG. 6F). The POLI fiber probes 100 reduced the tissue damage associated with the insertion of two separate electrodes (FIGS. 6G-6L).

Anatomical scans were acquired using a T2-weighted rapid acquisition with refocused echoes (RARE) pulse sequence with 18 1-mm thick slices with the field of view (FOV) of 20 mm×20 mm, the echo time (TE) of about 34.7 ms, and the repetition time (TR) of 2 seconds. Functional scans were performed using a T2*-weighted echo-planar imaging (EPI) sequence for detection of stimulus-induced BOLD contrast, with TE of 16 ms, TR of 2 seconds, FOV of 20 mm×20 mm, and image size of 40 mm×40 mm. The DBS (e.g., 60 Hz, 100 μA, 2 seconds ON, 48 seconds OFF, repeated for 30 cycles) applied via the electrodes 120 (e.g., CNT electrodes) within POLI fiber probes 100 resulted in greater increase in BOLD signal than identical stimulation protocol delivered via silver electrodes under continuous EPI scans (POLI fiber 11.0±1.4, silver electrodes: 7.6±0.7, n=4 rats/group, FIGS. 6M and 6N). As shown in FIG. 6N, the POLI fiber (left bar) produces a greater increase in BOLD signal compared to identical stimulation delivered via silver electrodes (right bar). Without being bound to a particular theory, this may be due to the masking of voxels with high BOLD signals adjacent to the silver electrodes, with the POLI fiber probe 100 mitigating susceptibility-induced artifacts.

To test the utility of POLI fiber probes 100 for FSCV recordings of DA, these POLI fiber probes 100 and the two 230 μm bipolar stainless-steel electrodes (MS303, Plastics One) were implanted into the NAc core and the ventral tegmental area (VTA) of a Sprague Dawley rat (FIG. 6O). The VTA is comprised of approximately 56% DA neurons in rats, and the release of DA from NAc-projecting VTA DA neurons is the hallmark of reward. Following 24 bi-phasic electrical pulses (2 ms, 300 μA) delivered in the VTA, robust phasic DA concentration release in the NAc core was observed (FIGS. 6P-6R). The DA concentration was extrapolated via a post-experiment electrode calibration procedure as described below. Evoked DA levels were consistent over 6 stimulation epochs separated by 5-minute intervals with an average current of about 3.07±0.32 nA, corresponding to a concentration of about 199±20.55 nM, which is comparable to prior reports.

### POLI Fiber as a Tool to Assess Drug-Induced Perturbations in the Reward Circuit

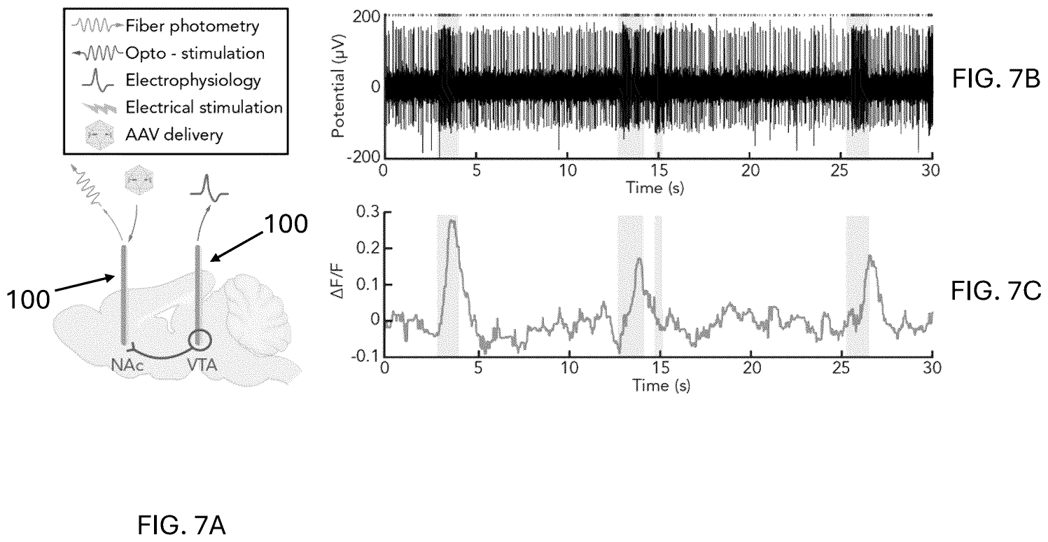

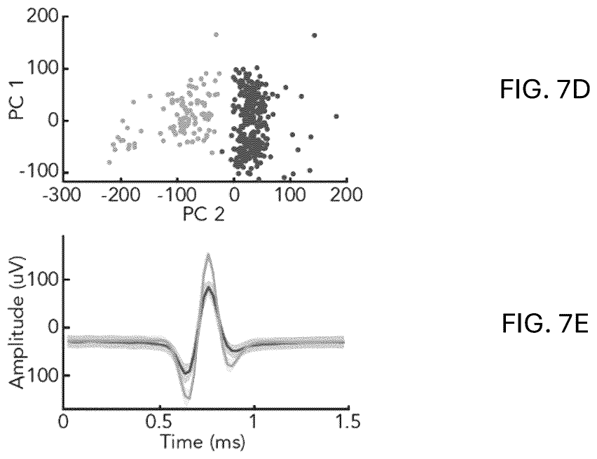

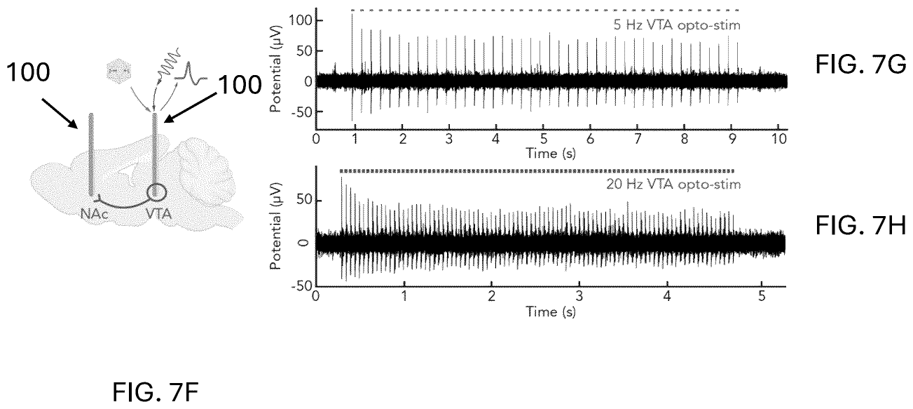

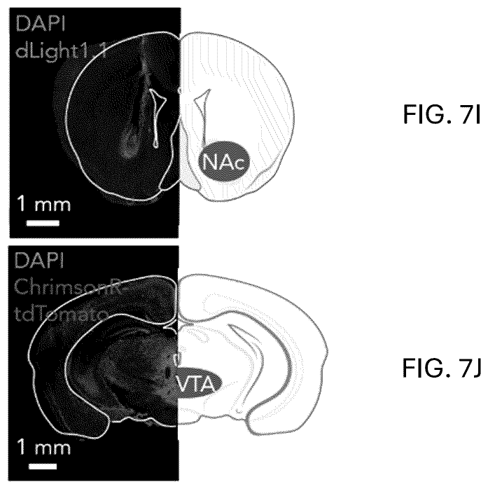

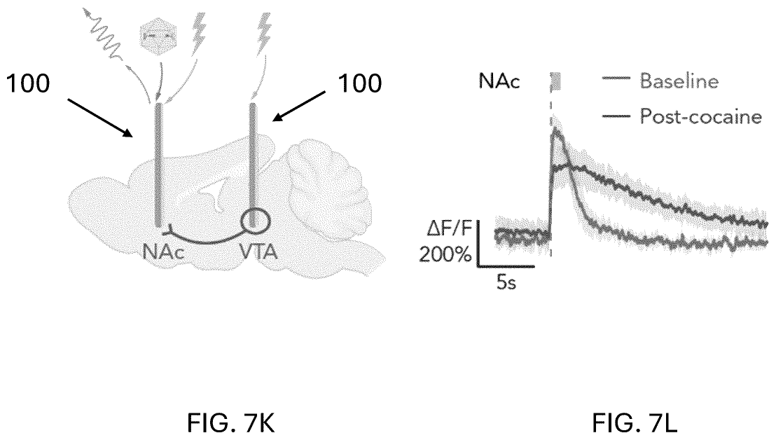

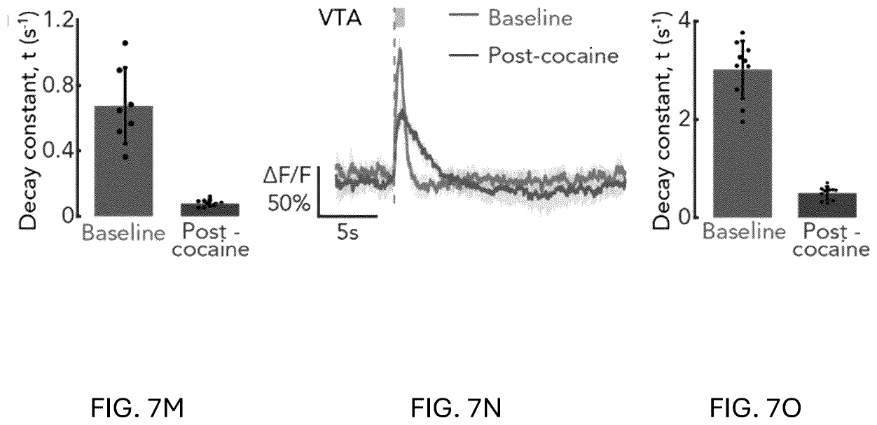

Maladaptive changes at key nodes of the mesolimbic reward circuit, for example, the VTA and NAc, accompany chronic drug and alcohol use. Developing tools to reveal changes in the VTA and NAc signaling in response to drugs of abuse holds the potential to advance the study and treatment of substance use disorders. Herein the utility of POLI fiber probes 100 to interrogate the DA projection circuit between the VTA and the NAc at baseline conditions as well as in the presence of cocaine, a well-characterized modulator of DA signaling in the brain, was evaluated. Adult transgenic DAT::Cre mice were implanted with POLI fiber probes 100 in the VTA and the NAc simultaneously (n=10, 5 male, 5 female). A fluorescent dopamine indicator dLight1.1 under pan-neuronal promoter human synapsin (hSyn) and packaged into an adeno-associated virus (AAV9) was delivered into the NAc via the microfluidic channel 110 of the POLI fiber probes 100 during implantation. The excitatory opsin ChrimsonR fused to a fluorescent protein mScarlet under CamkIIa promoter and packaged into an AAV9 vector was delivered into the VTA (FIG. 7A). Following 14 days of incubation, experiments including electrophysiological recordings in the NAc and VTA, photometric recording of dLight1.1 fluorescence in the NAc, optogenetic stimulation of DA neurons in the VTA, and electrical stimulation in the VTA or NAc were performed.

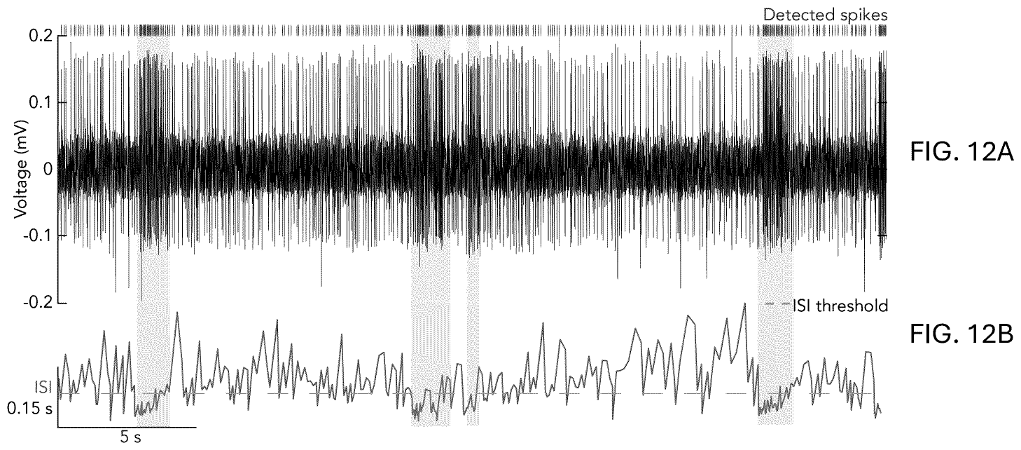

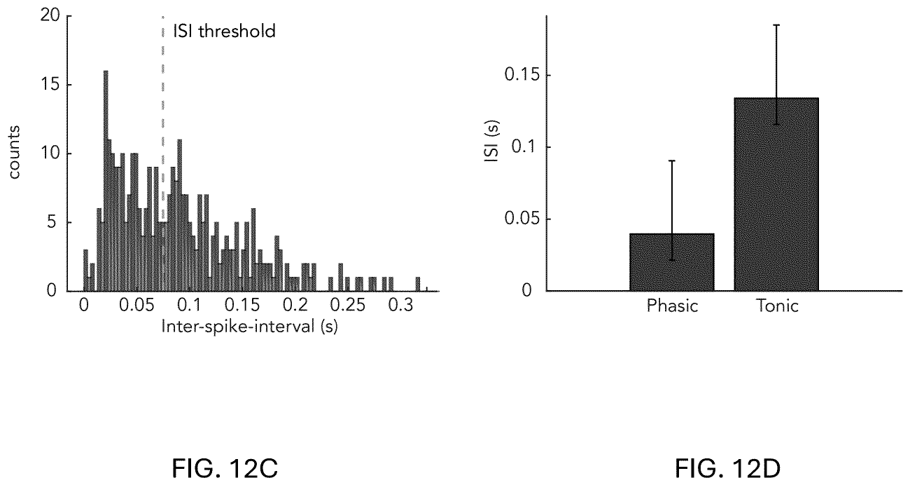

First, the spontaneous activity, including electrophysiology in the VTA and NAc with simultaneous photometry recording of DA dynamics via dLight1.1 photometry in the NAc (n=10 mice, 5 male, 5 female) was recorded. FIGS. 7B and 7C show example simultaneous electrophysiological recordings in the VTA and dLight1.1 photometry readings in the NAc. Principal components analysis and k-means clustering of the data in FIG. 7B revealed the spike waveforms of two putative neuronal units (FIGS. 7D and 7E). These VTA units exhibited tonic firing with an average rate of about 7.46 Hz, interspersed with bouts of high-frequency (e.g., about 25.2 Hz) phasic activity (FIGS. 7D and 12A-12D) which is characteristic of VTA DA neurons. Epochs of phasic firing in these VTA neurons preceded dLight1.1 fluorescence transients recorded in the NAc (FIGS. 7B and 7C), further suggesting their DA-producing identity.

Next, electrophysiological signals in the VTA were recorded while delivering 635 nm laser pulses (5 Hz and 20 Hz frequency; 10 ms pulse width) through the waveguide in the same POLI fiber probe 100 to drive DA neurons expressing ChrimsonR (FIG. 7F). Both 5 Hz and 20 Hz optogenetic stimulation drove activity of the VTA DA neurons (FIGS. 7G and 7H). Expression of ChrimsonR-mScarlet in the VTA and dLight1.1 in the NAc was corroborated with confocal microscopy in postmortem brain samples (FIGS. 7I and 7J).

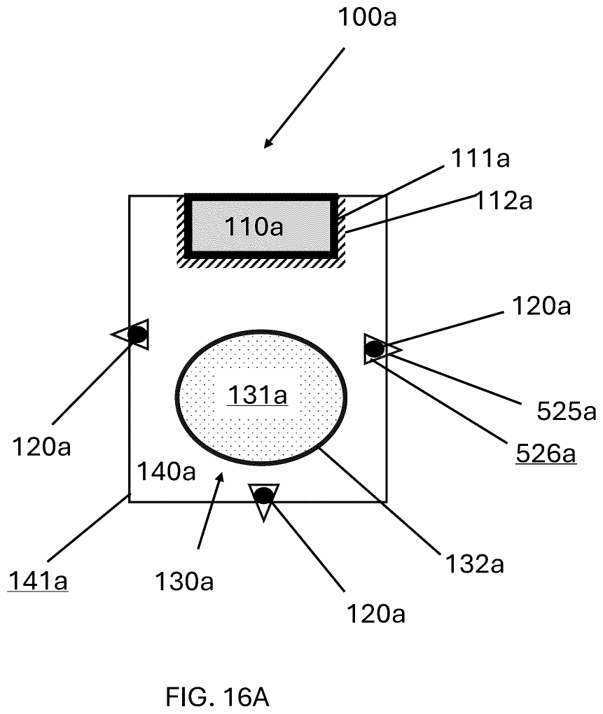

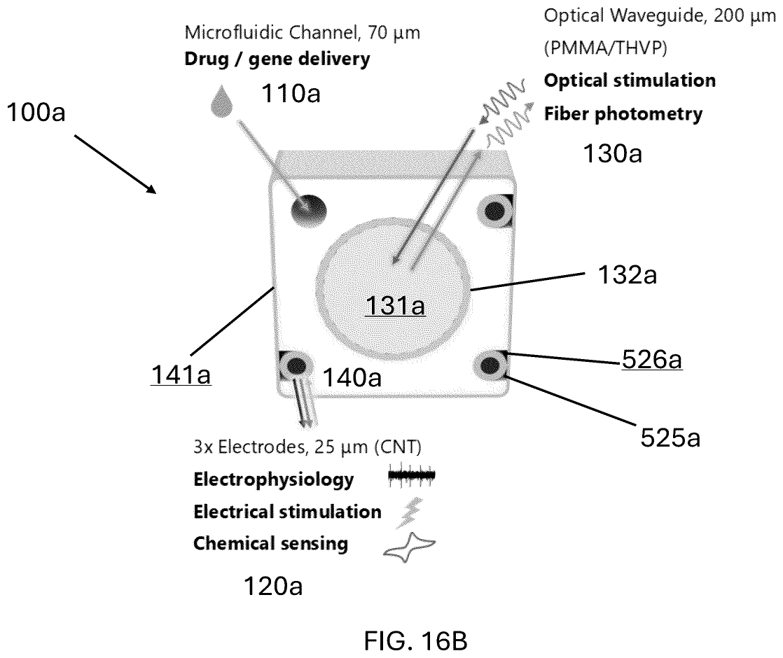

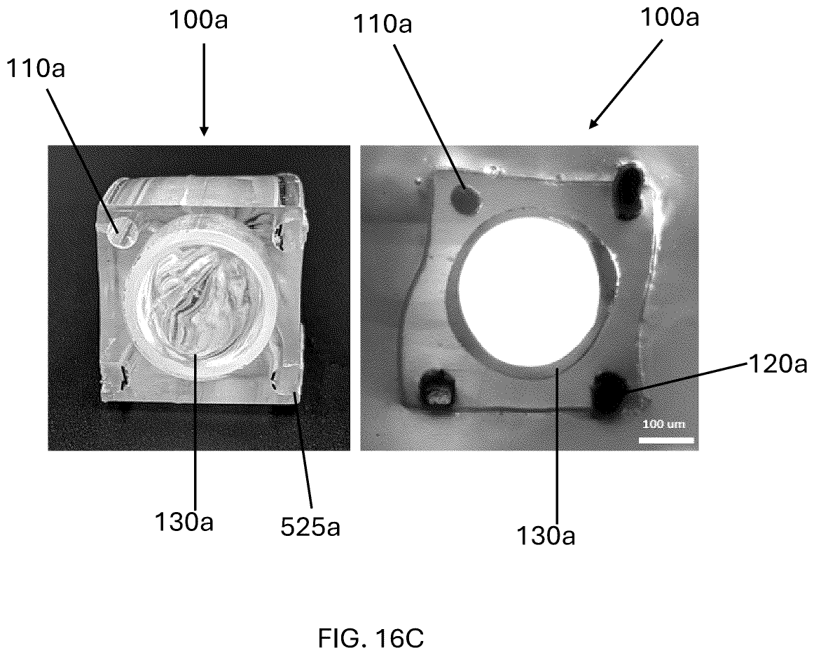

During the design and fabrication of the POLI fiber probe 100, another example POLI fiber probe, also referred to herein as a notch fiber or notch fiber probe, was made using post-draw integration of carbon nanotube (CNT) yarn rather than convergence during the fiber thermal drawing process. FIGS. 16A-16C illustrate a notch fiber probe 100a. The notch fiber probe 100a was made initially to test the concept in a cost-effective way, since convergence integration of six CNT microwires into the POLI fiber probe 100 fiber may require about >60 m of 20 μm-diameter CNT microwire (e.g., microwire 122). The notch fiber probe 100a may be made from the any of the components disclosed above for the POLI fiber 100.

Fabrication of the notch fiber 100a was nearly identical to that of the POLI fiber probe 100. The notch fiber 100a may include the same materials as the POLI fiber probe 100. For example, the notch fiber 100a may include an optical waveguide 130a with a core 131a and a cladding 132a. The core 131a may be poly(methyl methacrylate) (PMMA) and cladding 132a may be THVP (terpolymer of tetrafluoroethylene, hexafluoropropylene, and vinylidene fluoride) cladding, with the same dimensions as the POLI fiber probe 100. The notch fiber 100a may also include a microfluidic channel 110a with a wall 111a and an elastomer layer 112a. The notch fiber 100a may also include a housing 140a. The notch fiber 100a may include notched grooves 525a along three sides of the preform that were drilled in place of the six hollow channels (e.g., channels 121) found in the POLI fiber preform. These notches may be filled with an elastomer 526a (e.g., an SEBS elastomer (styrene ethylene butylene styrene)) to retain their shape during the drawing process.

After the notch fiber 100a was drawn, these strips of elastomer 526a (e.g., SEBS) (which may have a final width of approximately 5 μm to 50 μm, for example about 20 μm) may be peeled from the notches on the sides of fiber sections, and the electrodes 120a (e.g., a CNT microwire) may be pressed into these groves along the sides of the notch fiber 100a. For example, the notch fiber 100a may include three notches for three electrodes 120a. The notch fiber 100a may be coated with a coating 141a via chemical vapor deposition. The coating 141a may be about 2 μm to about 10 μm, for example about 5 μm of Parylene-C. The coating 141a may electrically insulate and fix the electrodes 120a (e.g., a CNT microwires) in place. The coating 141a may be deposited into the notched groves 525a and surround the electrodes 120a. The coating 141a may also be deposited over the entire notch fiber 100a. For example, the coating 141a may cover the microfluidic channel 110a, the electrodes 120a, the optical waveguide 130a, the housing 140a, and/or the notched grooves 525a. The coating 141a may be instead of or in addition to the housing 140a. This produced a final notch fiber 100a that is functionally equivalent to the POLI fiber probe 100, but with three electrodes 120a rather than six. The methods used to manufacture the notch fiber probe 100a may be used to incorporate CNT microwires (e.g., electrodes 120a) into a fiber probe without using fiber drawing and convergence.

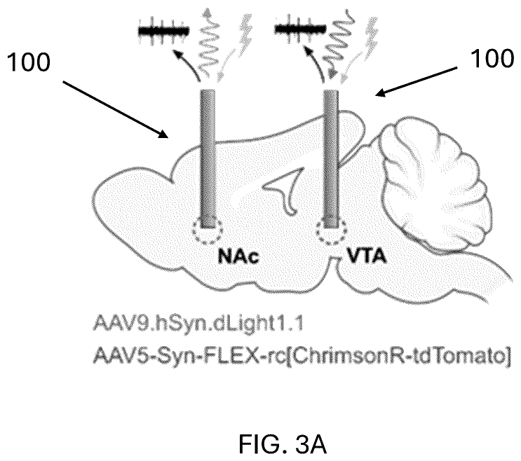

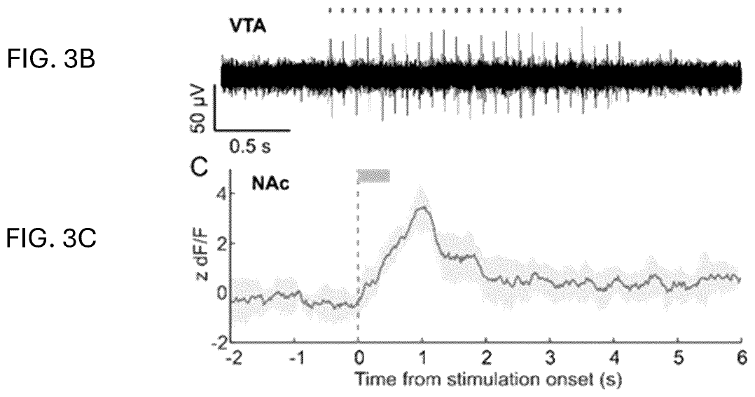

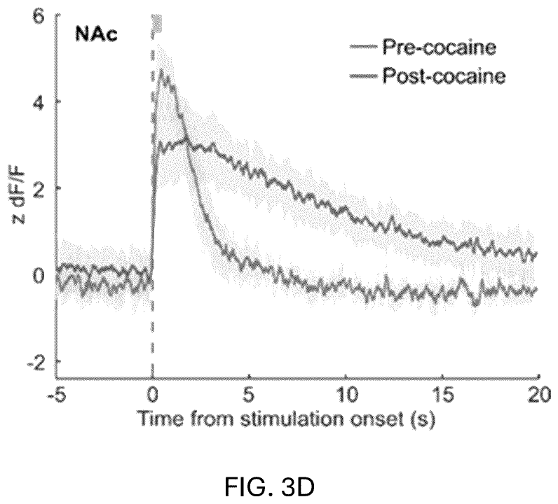

Cocaine is known to inhibit DA reuptake within the mesolimbic pathway. To determine whether cocaine-induced changes in DA dynamics could be resolved using recording and stimulation capabilities of the POLI fibers, these POLI fibers probes 100 or Notch fiber probes 100a—an example POLI fibers probe 100 were implanted into the NAc and VTA of C57BL/6J wild-type mice (n=5, male), with dLight1.1 delivered to the NAc as described above (FIG. 7K). Using the CNT electrodes in POLI fiber probes 100, neurons in the VTA were electrically stimulated (60 Hz; 2 ms biphasic pulses; 200 μA; 0.5 seconds ON, 30 seconds OFF) and dLight1.1 signals corresponding to stimulation-evoked DA were measured in the NAc, where DA neurons in the VTA send their projections, before and after cocaine administration (20 mg/kg, intraperitoneal) (FIG. 7K). A slower decay of stimulation-evoked dLight1.1 transients following cocaine injection was observed, indicating inhibited DA reuptake in the NAc (FIG. 7M) (see also FIGS. 3A-3D).

This experiment was repeated using Notch fibers probes 100a, with electrical stimulation this time applied directly to the axon terminals in NAc rather than to the cell bodies in the VTA. The stimulation-evoked DA transients recorded in the NAc were larger for direct NAc stimulation as compared to VTA stimulation, and cocaine administration caused a similar slowing of DA decay by inhibiting DA reuptake (FIG. 7L). In these experiments, it was shown that fiber-integrated CNT electrodes (e.g., electrodes 120a) could be used for electrical stimulation to evoke DA release in the NAc, either by stimulating the cell bodies in the VTA, or by directly stimulating the axon terminals in the NAc. Furthermore, integrated optical waveguide (e.g., waveguide 130a) could concomitantly record the DA dynamics via dLight1.1 photometry. Consistent with prior work, following administration of cocaine, a decrease in a rate of decay of stimulation-evoked dLight1.1 fluorescence stemming from the blocking of the dopamine transporter (DAT) was observed (FIG. 7N and 7O).

### Experimental Section

POLI Fiber Fabrication: Multifunctional fibers (e.g., POLI fiber probes 100) with six carbon nanotube microelectrodes (20 μm diameter) (e.g., electrodes 120 and/or 120), one 200 μm diameter PMMA/THVP polymer optical waveguide (e.g., optical waveguide 130 and/or 130), and one rectangular microfluidic channel (100 μm×40 μm lumen) (e.g., microfluidic channel 110) (FIG. 4D) were produced via a thermal fiber drawing process (FIG. 4A). As described above, the microfluidic channel 110 may be any suitable shape, including, but not limited to, rectangular, square, circular, or oval. For example, the microfluidic channel 110 may be rectangular in shape to maximize the cross-sectional area of the microfluidic channel 110 given the geometry of the POLI fiber probe 100. The microfluidic channel 110 may be about 25 μm×25 μm to about 300 μm×300 μm, for example about 100 μm×40 μm. Thermal drawing begins with fabricating a macroscale fiber preform. In this case, polycarbonate (PC) bars (McMaster-Carr, Impact Resistant Polycarbonate) were milled with a computerized CNC Mill to form the bulk structure of the preform with six 1.59 mm electrode channels, a 14.29 mm opening for the waveguide (e.g., optical waveguide 130), and a 10 mm×5 mm rectangular channel for the microfluidic channel (e.g., microfluidic channel 110). Separate PC parts were consolidated in a hot press with PTFE spacers used to maintain the openings for the electrode channels (e.g, channel 121 and/or 121), optical waveguide (e.g., optical waveguide 130 and/or 130), and fluidic channel (e.g., microfluidic channel 110 and/or 110) (185° C., 5 psi, 60 minutes).

The optical waveguide preform was fabricated by hot-pressing THVP pellets (3M Dyneon, THVP 2030GZ) into a 200 μm-thick film (160° C., 5 psi pressure, 60 minutes). This THVP film was then rolled tightly around a 12.7 mm diameter PMMA rod (US Plastic Corp., Clear Extruded Acrylic Rod) to a final diameter of about 14.3 mm. The waveguide preform was inserted into the central opening in the main PC preform, and they were consolidated in a vacuum oven at about 160° C. for about 40 minutes.

The microfluidic channel preform was produced by milling polycarbonate sheets and consolidating them into an about 3.55×8.52 mm rectangle in the hot press using an aluminum mold and Teflon spacer placed in the lumen (185° C., 5 psi, 30 minutes). This PC rectangle was then hot-pressed into a layer of Styrene-Ethylene-Butylene-Styrene (SEBS, Kraton G1657) in an aluminum mold to outer dimensions of about 4.97×9.94 mm (130° C., 5 psi, 10 minutes) to form the wall 111 and/or 111 and the elastomer layer 112 and/or 112, respectively. The PC/SEBS microfluidic preform was then placed into the rectangular channel on the side of the main PC preform and consolidated in the hot press (130° C., 5 psi, 10 minutes). As the final step to complete the multifunctional fiber preform, the corners of the preform were rounded with a 3.55 mm rounded corner end mill.

During the fiber drawing process, the preform was placed into a vertical cylindrical oven heated to 280° C. The carbon nanotube electrodes were incorporated into the fiber using convergence. Six spools of 20 μm diameter single-filament CNT fiber (Dexmat, Galvorn Fiber) were oriented above the thermal drawing oven and the ends of the CNT fibers were fed into the electrical channels of the preform (e.g., channels 121). As the preform was heated above the glass transition temperature, T_{g}, of the constituent polymers, it flowed downward and formed a neck, reducing the diameter of the preform. When the lower end of the preform reached a capstan situated below the oven, the lower end of the preform was cut off and the necking part of the fiber was fed into the capstan. The capstan speed, v_{capstan}, was slowly increased as the preform was fed into the oven at v_{feed}. The ratio between v_{capstan} and v_{feed} determined the reduction factor, or the ratio between the cross-sectional area of the preform and the fiber. Herein it was assumed that v_{feed}=0.25 mm/minute and v_{capstan}=1.26 m/minute for a reduction factor of 1:71 between the fiber and preform dimensions. As the fiber diameter decreased to the final target size of about 280 μm×310 μm, the electrode channels in the preform decreased to the size of the CNT wire until they tightly constricted around the wires and pulled them into the fiber at the same rate that the fiber was drawn at.

Polymer optical fibers fabrication: Three polymer optical fibers (PC/PMMA, COC/PMMA, PMMA/THVP) were fabricated using the thermal drawing process, using a process similar as the one outlined above. Briefly, a PC/PMMA optical waveguide was fabricated by rolling 0.05 mm PMMA films (GoodFellow, #ME301050) around a 12.7 mm PC rod (McMaster-Carr, #8571K14) until the assembly reached a diameter of about 13.3 mm, which was then consolidated under vacuum at a temperature of 170° C. for 40 minutes. A COC/PMMA optical waveguide was fabricated by first molding COC 6013 pellets (Ajedium) in a vacuum oven at 280° C. for 12 hours into an about 25.4 mm square bar, which was then lathed to form an about 16.4 mm rod. Then 0.05 mm PMMA films (GoodFellow) were rolled until the assembly reached a diameter of about 17.1 mm, before getting consolidated in a vacuum oven at a temperature of 170° C. for 40 minutes. Finally, a PMMA/THVP preform was fabricated by rolling an about 200 μm-thick THVP film (3M Dyneon THVP 2030GZ, processed as described above), around an about 19.05 mm PMMA rod (US Plastic Corp. Clear Extruded Acrylic Rod) until it reached about 19.8 mm in diameter, before being consolidated under vacuum at a temperature of 160° C. for 40 minutes. Each preform was transformed into a fiber (e.g., POLI fiber probe 100) using the thermal drawing process with a draw temperature of about 260° C. (PC/PMMA), about 215° C. (COC/PMMA), or about 180° C. (PMMA/THVP) until each fiber diameter reached about 400 μm.

Dual Fiber Device Assembly: Sections of the multifunctional fiber were assembled into a custom dual-fiber probe device (e.g., assembly 590), designed with precise spacing between the probes to enable simultaneous implantation into the mouse nucleus accumbens (NAc) and ventral tegmental area (VTA). First, the POLI fiber probe 100 was cut into 4 cm sections. The microfluidic channel 110 was then connected to tubing (e.g., tubing 250 and/or 550). The tubing 250 and/or 550 may be poly(vinyl chloride) (PVC) tubing (Tygon PVC, McMaster-Carr #8349T11). The microfluidic channel 110 may be connected to the tubing 250 and/or 550 by peeling the first approximately 20 mm of the microfluidic channel 110 from the side of the fiber, trimming it to approximately 3 mm in length, inserting this into the tubing 250 and/or 550, and sealing the junction with an epoxy (e.g., epoxy 260). The epoxy 260 may be an UV-curable epoxy (Norland Optical Adhesive 68).

Next, the housing 140 (e.g., the PC cladding) surrounding the optical waveguide 130 and the electrodes 120 (e.g., the microelectrode wires) was chemically etched from the upper portion of the POLI fiber probes 100, where the microfluidic channel 110 had already been separated, by submerging into dichloromethane (DCM, Sigma Aldrich L090000) for 2 minutes. The etch-exposed electrodes 120 (e.g., CNT microwires) were separated from the exposed portion of the polymer optical waveguide 130, and the waveguide 130 was coated with 5-minute epoxy (Devcon 14240), inserted into a 2.5 mm diameter stainless steel optical ferrule (e.g., optical ferrules 580) (Thorlabs #SF270-10), and allowed to cure. Excess length of the polymer optical waveguide 130 protruding out from the top of the ferrule 580 was trimmed, then the ferrule was polished using AlOx lapping films (e.g., Thorlabs LF5P, LF3P, LF1P, LF03P).

Next, two devices were inserted into a custom-designed printed circuit board (e.g., PCB 591) (PCB, PCBWay) by inserting the optical ferrules 580 through precisely spaced VIAs in the PCB 591. The layout of the PCB 591 may be custom-designed so that the PCB 591 may fit the relative placement and/or geometry of the optical ferrules 580, along with a thru via for connecting the microwires. The ferrules 580 were secured in place in the PCB 591 with UV-curable epoxy. Next, each electrode 120 (e.g., the CNT microwire) from the two POLI fiber probes 100 was fed through a plated through-hole VIA in the PCB 591 and connected with an electrode interface board (EIB) gold connector pin (e.g., pin 581) (e.g., an Open Ephys OEPS-7010). Each of the plated through-holes was routed to a surface-mounted Omnetics electrical connector (e.g., connector 582) (Omnetics A79042). Finally, a custom-designed 3D-printed casing (e.g., housing 592) was attached to the bottom of the PCB 591 to enclose all connections, with the two POLI fiber probes 100 protruding from the bottom of the assembly 590. The two POLI fiber probes 100 may be trimmed to the appropriate lengths with the help of a 3D-printed cutting mold to reach the target brain regions.

### Optical Waveguide Characterization

The optical loss coefficient of the PMMA/THVP polymer optical waveguide 130 in the POLI fiber probe 100 was measured using the cut-back method. Briefly, 12 cm sections of each fiber waveguide were cut and connected to stainless steel optical ferrules as described above. After polishing the ferrule termination, the POLI fiber probe 100 was connected via optical patch cord to a 470 nm fiber-coupled LED light source (Thorlabs M470F4) driven by an LED driver (Thorlabs LEDD1B) at a constant drive current. The light power emitted from the tip of the POLI fiber probe 100 was measured using a digital optical power meter and photodiode power sensor (Thorlabs PM100D, S120C). Optical power output was measured at the original 12 cm fiber length and then repeatedly after cutting off 1 cm length of fiber to a total of 10 times. This was repeated for three POLI fiber probe 100 samples of each core 131-cladding 132 configuration to yield an exponential relation between fiber length and optical power output. The optical loss coefficient was characterized using Equation 1:

$$α = \frac{10}{x} {log}_{10} ( \frac{P ( x )}{P ( 0 )} ) , \qquad ( Equation 1 )$$

where P(x) is the optical power output at length x, and P(0) seconds the optical power output at the shortest length measured (approximately 2 cm).

Numerical apertures of polished, ferrule-coupled waveguides were experimentally measured using the 470 nm fiber-coupled LED light source described above and a low-light CMOS camera (FLIR Blackfly, BFS-U3-200S6M-C). Each POLI fiber probe 100 was mounted on a micrometer-coupled mechanical translation stage. The profile of the output beam was captured at regular relative distances (e.g., Δ=0 mm, 1 mm, 2 mm, 3 mm, 4 mm, 5 mm) between the CMOS sensor and the fiber facet (FIG. 9A). Beam profiles were fitted to Gaussian curves and the lateral beam extent, d, for each relative fiber-sensor distance was calculated as the two-sided width of the fitted beam profile (FIG. 9B) at which the maximum signal intensity (L₀) had decreased to L₀×e⁻². NA values were calculated as the angular beam divergence, or increase in 1/e² beam diameter at each 4¿, using Equation 2:

$${NA}_{i} = n sin ( θ ) = sin ( \frac{d ( {Δ}_{i} ) - d ( {Δ}_{i - 1} )}{2 ( {Δ}_{i} - {Δ}_{i - 1} )} ) , \qquad ( Equation 2 )$$

where n indicates refractive index of the measuring medium (nₐᵢᵣ=1), and θ indicates the angle of beam divergence. For N=3 waveguides of each composition, final NA values were calculated as linear fits to beam divergence data and compared to theoretical or manufacturer-reported values (FIG. 9C). For custom-drawn polymer waveguides, theoretical numerical apertures (NA) were calculated from reported refractive indices of the core (n_{core}) and cladding (n_{cladding}) materials using Equation 3:

$$NA = \sqrt{{n}_{core}^{2} - {n}_{cladding}^{2}} . \qquad ( Equation 3 )$$

### Agarose Brain Synthesis and Fluorescein Injection

A 5-times concentrated (5×) Tris-borate-EDTA (TBE) solution was prepared by dissolving about 54 g of Tris(hydroxymethyl)aminomethane (TRIS) (17926, Thermo Scientific) and about 27.5 g of boric acid (B0394, Sigma-Aldrich) in about 20 mL of 0.5M pH 8 Ethylenediaminetetraacetic acid (EDTA) (AM9260G, Invitrogen) and about 900 mL of deionized water. 0.6% w/v Agarose LE (50-192-7938, Fisher Scientific) was dissolved in heated in 1X TBE. Then about 1500 mL of the dissolved 0.6% agarose gel was poured into a brain mold (B009S5SL90, Amazon) and allowed to cool overnight at room temperature. Following brain phantom gelation, 10 μl of fluorescein (AAL1325122, Fisher Scientific) dissolved in 1×TBE at a concentration of 10 μg/ml was injected into the agarose brain using an extended length pipette tip.

### Electrochemical Characterization

Electrochemical properties of the electrodes 120 (e.g., 20 μm-diameter CNT microelectrodes) at the POLI fiber probe 100 tip were evaluated in a three-electrode cell using a potentiostat (Gamry Instruments, Gamry Interface 1010e). A graphite rod was used as the counter electrode, an Ag/AgCl reference electrode (Sigma Aldrich) was used as the reference, and 10 mM phosphate buffered saline (PBS) at pH 7.4 (Fisher Scientific) was used as the electrolyte. Electrochemical impedance spectroscopy (EIS) was performed in a range of about 1 Hz to about 100 kHz with 10 m Vₚ₋ₚ AC driving voltage. Cyclic voltammetry (CV) was performed at a sweep rate of 50 m V/s. The water window limits of the CNT electrodes were determined by incrementally increasing the negative limit of the CV scan until water reduction was observed (beginning at about −1.5 V), then the positive limit of the CV scan until a linear, resistive behavior indicating water oxidation was observed (beginning at about +1.2 V). Cathodic charge storage capacity (CSC_{C}) was determined from CV scans from about −1.5 to about +1.2 V by taking the time integral of the cathodic current.

To measure the cathodic charge injection capacity (CIC_{C}), voltage transient testing was performed using chronopotentiometry with biphasic, charge-balanced current pulses with t_{c}=tₐ=250 μs and tᵢₚ=250 μs for currents ranging from 100 μA to 500 μA. The maximum cathodic potential (E_{mc}) was determined as the instantaneous voltage 10 μs after the end of the cathodic current pulse. E_{mc} values were plotted as a function of injected current amplitude, and the linear relation was determined to estimate the current limit at which the electrodes 120 would reach its cathodic limit, −1.5 V. CIC_{C} was defined as Equation 4:

$${CIC}_{c} = \frac{{I}_{lim} \times {t}_{c}}{GSA} , \qquad ( Equation 4 )$$

where Iₗᵢₘ is the cathodic current limit, t, is the cathodic pulse width, and GSA is the electrode geometric surface area.

### Microfluidic Channel Characterization

To characterize the performance of microfluidic channels (e.g., microfluidic channels 110) in converged POLI fiber probe 100 samples (N=3), saline was injected at 25, 50, or 75 nL/minute into the microfluidic channel (e.g., microfluidic channels 110) using a syringe pump system (e.g., a UMP3 Syringe pump and NanoFil syringe, World Precision Instruments). A capillary attached to the microfluidic outlet was visualized under a microscope, and images were continuously recorded with video capture. A small volume (approximately 1 μL) of mineral oil was withdrawn into the outlet capillary such that the oil-water interface could be visualized. The interface position was tracked during the infusion and used to report flow rate as a function of the known capillary diameter (e.g., about 250 μm or about 375 μm).

### Mechanical Characterization

The stiffness of converged POLI fiber probes 100 (N=3, 360-380×400-420 μm²) compared to a comparably sized silica fiber (400 μm diameter) was characterized with dynamic mechanical analysis (DMA, TA Instruments, Discovery Q850). DMA measurements were performed in single-cantilever mode over a 17.5 mm sample length at 37° C. using 10 μm vertical deflections.

### Dual-Site Implantation in Mesolimbic Pathway

These animal procedures were approved by the MIT committee on Animal Care and performed in accordance with the IACUC protocol 0121-002-24. The plasmid pAAV-hSyn-dLight1.1 was purchased from Addgene (#111066) and packaged into AAV9 serotype in-house to a titer of 7.7×10¹² vg/mL (protocol described below). The Cre-dependent red excitatory opsin pAAV-CamKIIa-ChrimsonR-mScarlet-KV2.1 in AAV9 serotype was purchased from Addgene (#124651-AAV9) with a titer of ≥5×10¹² vg/mL. Wild type C57BL/6J (The Jackson Laboratory, #000664) and DAT-Ires-Cre (The Jackson Laboratory, #006660) mice aged 8 weeks were used for the study, and were housed in a normal 12 hour light/dark cycle with standard chow diet and water ad libitum. All surgeries were performed under aseptic conditions. Mice were anesthetized with 1-2% isoflurane, placed on a heat pad in a stereotaxic head frame (Kopf Instruments), and injected subcutaneously with slow-release buprenorphine (ZooPharm, 1.0 mg kg⁻¹). Ophthalmic ointment (Puralube) was applied to the animal's eyes to retain moisture. A midline incision was performed along the scalp, then the skull was repositioned by aligning and levelling Lambda and Bregma landmarks. Implantation and injection coordinates were established following the Mouse Brain atlas by Paxinos and Franklin as follows: ventral tegmental area (VTA; ML +0.45, AP −3.3, DC −4.3) and the nucleus accumbens (NAc; ML +1.25, AP +1.2, DV −4.3).

The dual-fiber device (e.g., assembly 590) was positioned above the animal's head and aligned to the stereotaxic frame using a custom-designed 3D-printed holder to enable accurate positioning. The microfluidic channels 110 of each POLI fiber probe 100 were connected to a NanoFil Syringe and UMP3 Microinjection pump (World Precision Instruments) via flexible poly(vinyl alcohol) tubing (e.g., tube 250 and/or 550), and the entire line was primed with sterile phosphate buffered saline, with care taken to eliminate any air bubbles. Craniotomies were performed using a rotary tool (Dremel Micro 8050) and a carbon steel burr (Heisinger, 19007-05) at each implantation target, with two additional craniotomies drilled over the contralateral cortex for placement of skull screws to serve as the reference and ground electrodes for electrophysiology recordings. The two stainless steel skull screws (McMaster-Carr #90910A310) attached to the dual-fiber device (e.g., assembly 590) were fixed to the skull with a T1 torx screwdriver such that the tip of each screw contacted the cortical surface. Solutions of viral vectors were then loaded into the microfluidic channel (e.g., microfluidic channels 110) of each POLI fiber probe 100 as follows: 500 nL of AAV9-dLight1.1 was drawn into the tip of the NAc-targeted POLI fiber probe 100 and 500 nL of AAV9-ChrimsonR was drawn into the tip of the VTA-targeted POLI fiber probe 100. The dual fiber device (e.g., assembly 590) was then lowered into the brain to the coordinates listed above.

Following implantation, viruses were infused from each fiber as follows: 250 nL of virus was injected at a rate of 50 nL/min. The assembly 590 was then cemented to the skull using 3 layers of C&B-Metabond adhesive acrylic (Parkell) followed by dental cement (Jet Set-4) to cover the base of the device and the skull screws. The mouse was subcutaneously injected with carprofen (e.g., about 5 mg kg⁻¹) and sterile Ringer's solution (e.g., about 0.6 mL) prior to being returned to the home cage, placed partially on a heating pad. Post-implantation, animals were provided with food and water ad libitum, were monitored for 3 days for signs of overall health, and were provided with carprofen injections (e.g., 0.6 mL, 0.25 mg mL⁻¹ in sterile Ringer's solution) as necessary.

### Functional In Vivo Experiments

In vivo experiments were performed a minimum of 14 days post-implantation to allow the dLight1.1 indicator and ChrimsonR opsin sufficient time for expression. Animals were anesthetized with about 1-2% isoflurane, placed on a heat pad, and ophthalmic ointment (Puralube) was applied to the animal's eyes. For fiber photometry and optogenetic stimulation experiments, the Neurophotometrics FP3002 system was used, and for electrical stimulation and electrophysiology experiments the Ripple Grapevine Scout with Pico+Stim headstage was used, and these systems were coupled with digital I/O to enable time-syncing. For ChrimsonR optogenetic stimulation trials, a 635 nm laser from the FP3002 system was used to deliver 10 mW/mm² optical power from the tip of the POLI fiber probe 100 in 10 ms pulses at 5 Hz or 20 Hz frequencies, with 60 seconds between pulse trains. The laser stimulation parameters were controlled from a MATLAB API script controlling the Ripple Grapevine system, via a BNC digital output to the FP3002 to drive the laser, such that a digital output timestamp was captured in the electrophysiology recordings each time a laser pulse was delivered.

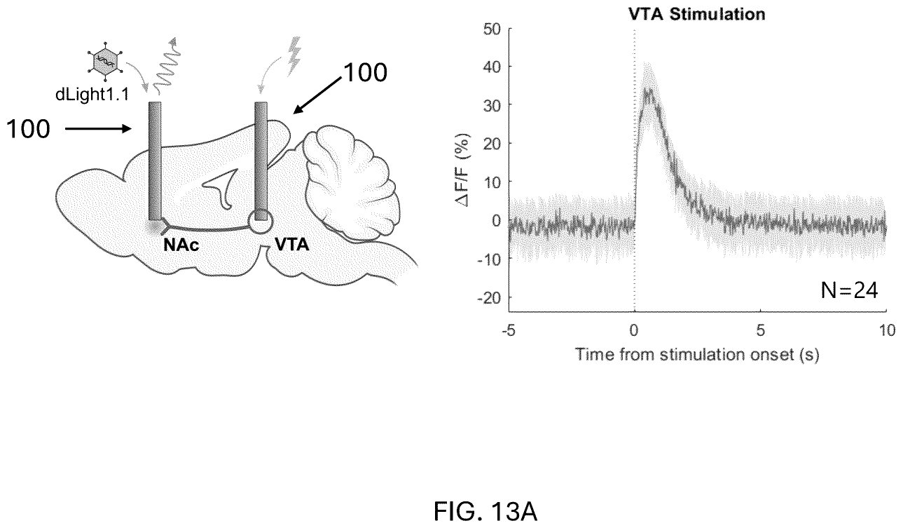

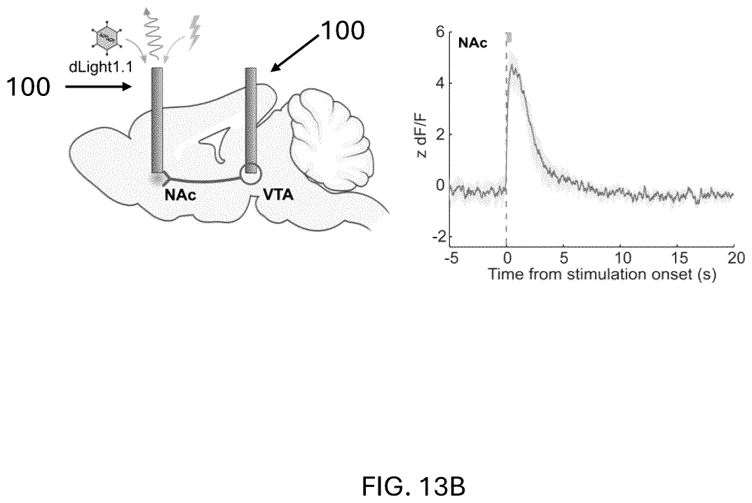

Electrophysiology signals were recorded at a sampling rate of 30 kHz. For fiber photometry recordings, the 470 nm (dLight/GCaMP) and 415 nm (isosbestic) LEDs in the FP3002 system were each calibrated to deliver 100 μW of optical power out of the tip of the POLI fiber probe 100, and recordings were performed at a 130 Hz sampling rate (e.g., about 65 Hz per wavelength alternating between 470 nm and 415 nm). The patch cord was photobleached for 1 hour prior to each photometry recording experiment. At the start of each photometry recording, a TTL pulse was sent via BNC digital output to the Ripple Scout system to enable time-syncing between the photometry recordings and the electrophysiology data. In trials where electrical stimulation was delivered during fiber photometry recording, a 5-minute baseline photometry signal was recorded prior to initiating stimulation, and TTL pulses were sent from the Ripple Scout system and recorded along with the photometry data to enable time-syncing. Electrical stimulation was applied as biphasic, charge-balanced, cathodic first pulses from a CNT electrode (e.g., electrodes 120) on the POLI fiber probe 100, with a skull screw as GND/return. FIGS. 13A and 13B show photometry recordings in the VTA (FIG. 13A) and the NAc (FIG. 13B) following implantation with two POLI fibers 100 targeting the NAc and the VTA.

In all experiments with electrical stimulation applied to evoke DA release, the following stimulation parameters were used based on previous literature: 60 Hz, 2 ms/phase biphasic, 200 μA, 0.5 second stimulation trains, 30 second interval between stimulation trains. In the experiments probing DA dynamics before and after cocaine exposure, fiber photometry recordings of dLight1.1 were performed while electrical stimulation pulses were delivered to the VTA or NAc (only one region stimulated per animal). A baseline photometry signal was recorded for 5 minutes before stimulation was initiated. Five stimulation pulse trains (60 Hz, 200 μA, 2 ms/phase biphasic, 0.5 second stimulation trains, 60 second interval) were delivered, then stimulation was paused and an intraperitoneal dose of cocaine (e.g., about 20 mg/kg) solution was injected. Five minutes after the IP injection of cocaine, electrical stimulation was restarted using the same parameters and 5 trains of stimulation were delivered.

### Fiber Photometry In Vivo

Ten Thy1-GcaMP6s mice (The Jackson Laboratory #024275, 8-weeks old) were implanted with either 400 μm silica waveguide (0.5 NA, Thorlabs; FP400URT, n=6 mice), or with 200 μm or 400 μm PMMA/THVP waveguide (n=1 mouse, and n=3 mice respectively). Using the same surgical approach as outlined above, each animal was implanted with an optical waveguide (e.g., optical waveguide 130) targeting their somatosensory barrel cortex (SIBF; ML ±3 mm, AP-1.2 mm, DV-0.4 mm). Following a week-long recovery, the animals were anesthetized using a ketamine/xylazine cocktail (ketamine, 100 mg/kg; xylazine, 10 mg/kg). Fiber photometry was performed using the same experiment approach as outlined above. Calcium signals were expressed as z-score. Following 2 minutes of baseline recording, whiskers contralateral to implanted SIBF were mechanically stimulating using a cotton tipped applicator, following a brushing motion. This stimulation was repeated every 30 seconds for a total of 7 times.

### Data Analysis

Electrophysiology and fiber photometry data were analyzed in MATLAB. Fiber photometry data were processed as follows: data were low-pass filtered below 25 Hz using a 2^{nd} order Butterworth filter with zero phase distortion. The first 2 minutes of the baseline recording were discarded to eliminate the effects of fast photobleaching that occurred at the start of the recording. The 470 nm and 415 nm signals were fit with a biexponential decay function to approximate the photobleaching dynamics, and the fitted photobleaching curves were subtracted from each signal to produce 470 nm and 415 nm signals that were corrected for photobleaching. The bleach-corrected 415 nm signal was regressed onto the bleach-corrected 470 nm signal with robust non-negative linear regression, thus scaling the isosbestic signal onto the 470 nm signal. This scaled isosbestic signal was then subtracted from the 470 nm bleach-corrected signal, producing a signal that has been corrected for both photobleaching and motion artifacts as recorded by the 415 nm isosbestic. Baseline fluorescence F0 was then calculated as the mean fluorescence value of the corrected 470 nm signa, during the baseline period. ΔF/F was finally calculated as ΔF/F=(corrected 470 nm signal−F0)/F0. Electrophysiology data were filtered into the spikeband from 300 Hz-6 kHz using a 2^{nd} order Butterworth bandpass filter with zero phase distortion. To extract and sort neuron spike units, spike waveforms were detected by threshold crossings, the waveforms were extracted and aligned by the spike peak, principal components analysis (PCA) was applied to the extracted waveforms, and k-means clustering was applied to cluster the spike waveforms and sort them into different neuronal units.

### AAV Packaging

AAV9-hSyn-dLight1.1 was produced following a previously reported protocol. Briefly, pAAV2/9n (Addgene #112865, http://n2t.net/addgene: 112865), pAAV-hSyn-dLight1.1 (Addgene #111066, http://n2t.net/addgene: 111066), and pHelper (CELL BIOLABS, INC.) were used for PEI-mediated triple transfection of HEK293T cells. The culture medium was collected at 72 hours and 120 hours, and the cells were harvested at 120 hours post-transfection, followed by purification by ultracentrifugation. The AAVs were collected in Dulbecco's phosphate-buffered saline (DPBS) (Gibco) with 0.001% Pluronic F-68 (Gibco). AAV titers were determined using a Taraka Bio AAV real-time PCR titration kit (#6233).

### Histology

Wild-type or DAT::Cre mice were implanted in the VTA and NAc POLI fiber probe 100 or commercial silica waveguides (300 μm, FT300UMT Thorlabs) were anesthetized with isoflurane, injected intraperitoneally with Fatal-Plus (100 mg kg⁻¹), and transcardially perfused with 50 mL of ice-cold PBS followed by 50 ml of ice-cold 4% paraformaldehyde (PFA) in PBS. The devices were carefully explanted, and the brains were removed and additionally fixed in 4% PFA in PBS for 24 hours at 4° C., then stored in PBS afterwards. Coronal slices (e.g., about 40-μm thick) were prepared using a vibratome (Leica, VT1000S) and a razor blade (Electron Microscopy Sciences, 72002) in ice-cold PBS. The slices were then stored in PBS at 4° C. in the dark until staining. Slices were permeabilized with 0.3% v/v Triton X-100 and blocked with 3% bovine serum albumin in PBS for 30 minutes. Slices were incubated overnight at 4° C. in the blocking solution with primary antibody (Iba1: rabbit anti-Iba1, ab178846 Abcam, 1:200 dilution; GFAP: Goat anti-GFAP, ab53554 Abcam, 1:200 dilution; CD68: mouse anti-CD68, ab31630, Abcam, 1:200 dilution; GFP: rabbit anti-GFP, A-11122 Invitrogen 1:200 dilution). Following incubation, slices were washed three times with PBS. The slices were then incubated with a secondary antibody (Donkey anti-Goat Alexa Fluor 555, A32816, Thermofisher, 1:1,000 dilution; Goat Anti-Rabbit Alexa fluor 488, ab150077, Abcam, 1:1,000 dilution; Goat Anti-Mouse Alexa Fluor 647, ab150115, Abcam, 1:1,000 dilution) for 1 hour at room temperature on a shaker followed by an additional three washes with PBS. Slices were then incubated with DAPI (4′6-diamidino-2-phenylindole) (1:20,000) for another 20 minutes, and washed three times with PBS. Fluoromount-G (SouthernBiotech) was used for mounting slices onto glass microscope slides (48311-703, VWR) and covered with No. 1.5 coverslips (CLS-1764-2250, Erie Scientific). A white light laser scanning confocal microscope (Stellaris 5, Leica) was used for imaging with a 10× objective. Regions of interest were chosen based on the implant locations.

### Surgery for MRI

These animal procedures were conducted in accordance with National Institutes of Health guidelines and with the approval of the MIT Committee on Animal Care with the with the IACUC protocol number 0721-059-24. The MRI experiments were performed with female Sprague-Dawley rats, age 8-12 weeks (Charles River Laboratories, Wilmington, MA). Eight rats were used for in vivo imaging experiments.

For stereotactic surgery, rats were anesthetized with isoflurane (3% for induction, 1.5% for maintenance) and placed on a water heating pad from Braintree Scientific (Braintree, MA) to keep body temperature at about 37° C. After stereotaxic frame fixation and topical lidocaine application a 3 cm lateral incision extending from bregma to lambda was made to expose the skull. Craniotomies (0.5 mm) were drilled unilaterally over the right Nucleus accumbens area (NAc), 6.0 mm anterior 1.7 mm and 3.5 mm lateral to bregma. After 30 minutes, POLI fiber probe 100 or a standard setup with custom-made silver bipolar electrode (two silver wires with each: inner diameter: 0.127 mm; outer diameter 0.178 mm, twisted together, A-M Systems, #786000) and a 200 μm fiber optic cannula (CFMXC10, Thorlabs) was lowered to 7.5 mm below the surface of the skull through the craniotomy and held in place by the stereotactic arm. The implants were fixed to the skull with dental cement (Secure Resin Cement, Parkell). Rats were allowed to recover for 8 weeks until the start of imaging experiments.

### MRI Imaging and Stimulation

Rats were anesthetized with isoflurane (induction 3% and maintenance 0.5% via nose-cone) and dexmedetomidine (0.05 mg/mL) for imaging. High field MRI acquisition was performed with a 20 cm-bore 9.4 T Bruker small animal scanner. A custom-made 30 mm single surface coil was used as a transceiver. Field inhomogeneity was minimized via MAPSHIM protocol in Paravision 6.0.1 software. Anatomical scans were acquired using a T₂-weighted rapid acquisition with refocused echoes (RARE) pulse sequence with 18 slices of 1 mm thickness, 20 mm×20 mm field of view (FOV), image size 200×200, echo time (TE) 34.7 ms, repetition time (TR) 2 seconds, and 8 averages. Functional scans were performed using T2*-weighted EPI sequence for detection of stimulus-induced BOLD contrast, with following parameters: TE of 16 ms, TR of 2 s, FOV 20 mm×20 mm, image size 40×40, 18 slices with slice thickness of 1 mm.

Deep brain stimulation (DBS) of 0.1 mA for 2 seconds at a frequency of 60 Hz was applied preceded by a 10-second baseline scan and followed by a 48-second resting scan combining into 1 cycle. 30 cycles were applied under continuous functional EPI scans.

### MRI Data Processing and Analysis

Images were reconstructed using the Para Vision 6.1 software and further processed with the National Institute of Health AFNI software package. Functional imaging time series were preprocessed in following steps: Slice timing correction, motion correction by a least-squares rigid-body volume registration algorithm, voxel-wise intensity normalization, spatial smoothing with Gaussian spatial kernel of 0.5-mm full-width at half-maximum and spatial resampling, all steps were performed in AFNI. High-resolution anatomical images of each animal were registered to a Waxholm coordinate space rat brain atlas. Functional images were aligned to anatomical images. After averaging 30 cycles of DBS, a maximum signal amplitude during an interval of 6 seconds after stimulation onset was determined and compared to an averaged signal during the preceding baseline interval via student-t-test to evaluate statistical significance of Z-values. To count the number of voxels in each individual with signal loss due to the implants, a threshold of SNR <5 was applied in the slice where the implant was visible on the anatomical images in the right hemisphere.

### FSCV Chemicals and Materials

Sodium chloride, sodium sulfate, calcium chloride, sodium phosphate monohydrate, and paraformaldehyde were purchased from Sigma-Aldrich (St. Louis, MO). Potassium chloride and magnesium chloride were purchased from Thermo Fisher Scientific (Waltham, WA). Dopamine (DA) hydrochloride was dissolved in 0.1 M HClO₄ to create a 10 mM DA stock solution. Phosphate buffered saline (PBS; (131.25 mM NaCl, 3.00 mM KCl, 10 mM NaH₂PO₄, 1.2 mM MgCl₂, 2.0 mM Na₂SO₄, and 1.2 mM CaCl₂)) was used at pH 7.4 to dilute the DA stock solution to 1.0 μM. Deionized water (EMD Millipore, Billerica, MA) was used to prepare all aqueous solutions.

### FSCV and Electrochemical Measurements

The POLI fiber probe 100 were conditioned by surface polishing using a KT Brown micro-pipette beveller (model BV-10, Sutter Instruments, Novato, CA). Before each use, each POLI fiber probe 100 was inserted into a glass capillary (A&M Systems, Inc., Carlsborg, WA) previously pulled using a vertical PE-22 Electrode Puller (Narishige, Tokyo, Japan) to a diameter of 500 μm, exposing 1 mm of the entire POLI fiber probe 100 length. The capillary was used to backfill with 1M KCL and attach the POLI fiber probe 100, ensuring a proper connection with the electrode holder (Warner Instruments, Holliston, MA). The tips were sealed by using 5-minute epoxy (J-B weld, Sulphur Springs, TX), and the silver wire in the electrode holder was connected through the backfilled capillary. All solutions were injected into a flow cell from a syringe at a rate of 2 mL min⁻¹ by a pump (Harvard Apparatus, Holliston, MA). The injection and analyte-buffer mixing was facilitated with a six-port, stainless steel air actuator (VICI Valco Instruments, Houston, TX). DA voltammograms were obtained using a Chem-Clamp potentiostat (Dagan Corp., Minneapolis, MN) coupled to a UNC breakout box (UNC Electronics Shop, Chapel Hill, NC) with a 1 MΩ head stage. HDCV software (UNC at Chapel Hill) was used for data acquisition and analysis. Ag/AgCl wires were used as reference electrodes. A 3 kHz low-pass filter was used to detect DA with a triangular waveform that scanned from about −0.4 V to about 1.3 V and back at 100 V/s at 10 Hz.

### FSCV Animal Experiments In Vivo

These animal experiments were performed as approved by the Animal Care and Use Committee (ACUC) of the University of Virginia. Male Sprague Dawley rats (Charles River Laboratories, Wilmington, MA, USA) between 280-320 g were anesthetized with urethane (0.3 ml/100 g, 5% saline solution, IP). A local anesthetic (bupivacaine) was used on exposed skin and muscle tissue during surgery. The rat was placed in a stereotaxic frame, and craniotomies were drilled precisely to place the stimulating and working electrodes according to the atlas of Paxinos and Watson. The POLI fiber probe 100 was lowered into the NAc core (+1.3 mm AP, +2.0 mm ML, −7.1 mm DV), and a bipolar stimulating electrode (Plastics One, Roanoke, VA, USA) was lowered into the VTA (−4.7 mm AP, +0.9 mm ML, −8.5 mm DV). DA was detected using FSCV at the NAc following stimulation in the VTA with 24 biphasic electrical pulses (2 ms, 300 μA, 60 Hz) delivered every 5 minutes. The dorsoventral coordinate of the electrodes in the VTA was adjusted to maximize the DA release. DA release in the NAc core was for 30 minutes. The recording electrodes were then subject to post-experiment calibration. Following in vivo measurements, a solution of 2.0 μM dopamine in PBS was used to recalibrate the linear relationship used to correlate peak current values detected in vivo to DA concentration.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the components so conjoined, i.e., components that are conjunctively present in some cases and disjunctively present in other cases. Multiple components listed with “and/or” should be construed in the same fashion, i.e., “one or more” of the components so conjoined. Other components may optionally be present other than the components specifically identified by the “and/or” clause, whether related or unrelated to those components specifically identified. Thus, as a non-limiting example, a reference to “A and/or B”, when used in conjunction with open-ended language such as “comprising” can refer, in one embodiment, to A only (optionally including components other than B); in another embodiment, to B only (optionally including components other than A); in yet another embodiment, to both A and B (optionally including other components); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of components, and, optionally, additional unlisted items. Only terms clearly indicated to the contrary, such as “only one of” or “exactly one of,” or, when used in the claims, “consisting of,” will refer to the inclusion of exactly one component of a number or list of components. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more components, should be understood to mean at least one component selected from any one or more of the components in the list of components, but not necessarily including at least one of each and every component specifically listed within the list of components and not excluding any combinations of components in the list of components. This definition also allows that components may optionally be present other than the components specifically identified within the list of components to which the phrase “at least one” refers, whether related or unrelated to those components specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including components other than B); in another embodiment, to at least one, optionally including more than one, B, with no A present (and optionally including components other than A); in yet another embodiment, to at least one, optionally including more than one, A, and at least one, optionally including more than one, B (and optionally including other components); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A multifunctional fiber comprising:
a microfluidic conduit to deliver at least one of a drug, a gene, or a chemical to a mammalian subject;
at least one electrode to enable at least one of electrical recording of neural activity, electrochemical recording of neural activity, or electrical stimulation of neural activity in the mammalian subject; and
an optical waveguide to enable at least one of optogenetic stimulation or fiber photometry,
wherein the microfluidic conduit is peelable from the multifunctional fiber.

2. The multifunctional fiber of claim 1, wherein the optical waveguide comprises at least two polymers and wherein the at least two polymers comprise at least one of poly(methyl-methacrylate) (PMMA)/Cyclic olefin copolymer (COC), PMMA/Polycarbonate (PC), or PMMA/tetrafluoroethylene hexafluoropropylene, vinylidene fluoride (THVP).

3. The multifunctional fiber of claim 1, wherein the optical waveguide comprises a core comprising poly(methyl-methacrylate) and a cladding comprising tetrafluoroethylene, hexafluoropropylene, and vinylidene fluoride.

4. The multifunctional fiber of claim 1, wherein the multifunctional fiber is formed from thermal drawing.

5. The multifunctional fiber of claim 1, wherein the multifunctional fiber further comprises at least one layer of an elastomer between the microfluidic conduit and the multifunctional fiber.

6. The multifunctional fiber of claim 1, wherein the at least one electrode enables the electrical recording of neural activity, the electrochemical recording of neural activity, and the electrical stimulation of neural activity in the mammalian subject concurrently.

7. The multifunctional fiber of claim 1, wherein the electrochemical recording of neural activity is a chemical recording of a neurotransmitter using fast-scan cyclic voltammetry.

8. The multifunctional fiber of claim 7, wherein the neurotransmitter is dopamine.

9. The multifunctional fiber of claim 1, wherein the at least one electrode has a charge injection capacity of about 5 mC/cm² to about 30 mC/cm².

10. The multifunctional fiber of claim 1, wherein the at least one electrode has a cathodic charge storage capacity of about 5000 mC/cm² to about 9000 mC/cm².

11. The multifunctional fiber of claim 1, wherein the electrical stimulation is deep brain stimulation.

12. The multifunctional fiber of claim 1, wherein the at least one electrode comprises at least one of a carbon nanotube (CNT) fiber, a tungsten (W) microwire, or a conductive microwire.

13. The multifunctional fiber of claim 1, wherein an end of the microfluidic conduit is peeled back from the multifunctional fiber and further comprising a tube coupled to the end of the microfluidic conduit.

14. The multifunctional fiber of claim 13, wherein the end of the microfluidic conduit is inserted into a lumen of the tube.

15. The multifunctional fiber of claim 13, wherein the tube is mechanically connected to the multifunctional fiber with an epoxy.

16. An assembly comprising the multifunctional fiber of claim 1, the assembly comprising:
a housing to hold a proximal end of the multifunctional fiber, wherein a distal end of the multifunctional fiber is configured to be interested into tissue and a proximal end of the microfluidic conduit is peeled back from the multifunctional fiber;
a tube coupled to the proximal end of the microfluidic conduit;
a printed circuit board operably connected to the at least one electrode; and
an optical ferrule operably connected to the optical waveguide.

17. A method of making a multifunctional fiber probe assembly, the method comprising:
forming a polymer preform defining at least a microfluidic channel, an optical waveguide channel, and an electrode channel;
thermally drawing the polymer preform to form a polymer fiber;
converging at least one electrode microwire through the electrode channel; and
mechanically separating one end of the microfluidic channel from the polymer fiber.

18. The method of claim 17, wherein forming the polymer preform defining the microfluidic channel further comprises:
forming a first polymer wall around the microfluidic channel; and
forming a second polymer layer around the first polymer wall, wherein the second polymer layer has a weak adhesion to the first polymer wall.

19. The method of claim 17, further comprising:
inserting the microfluidic channel into a lumen of a tube; and
sealing the tube to the polymer fiber with an epoxy.

20. The method of claim 19, further comprising:
exposing a distal portion of the electrode microwire running through the electrode channel;
electrically coupling the distal portion of the electrode microwire to a printed circuit board;
mechanically coupling the optical waveguide channel to an optical ferrule; and
inserting a proximal end of the polymer fiber into a housing.
